(19) United States
(12) Patent Application Publication
(10) Pub. No.: US 2025/0195167 A1
(43) Pub. Date: Jun. 19, 2025

(54) ERGONOMIC SURGICAL ROBOTIC SYSTEM

(71) Applicant: PROCEPT BioRobotics Corporation, San Jose, CA (US)

(72) Inventors: Mark BAERENRODT, Woodside, CA (US); Yucheng ZHANG, San Jose, CA (US); Peter BENTLEY, Reno, NV (US); Angela ZHOU, Ramon, CA (US); Lewis CHEN, Mountain View, CA (US); Kee Sook JEON, Petaluma, CA (US); Lynne SMITH, St. Louis, MO (US)

(73) Assignee: PROCEPT BioRobotics Corporation, San Jose, CA (US)

(21) Appl. No.: 18/539,023

(22) Filed: Dec. 13, 2023

(51) Int. Cl. A61B 50/20; A61B 8/00; A61B 8/12

(52) U.S. Cl. CPC A61B 50/20; A61B 8/12; A61B 8/40; A61B 8/469; A61B 8/4488

(57) ABSTRACT

A user interface comprising a touch screen display can be placed above a patient. An arm allows a user to place the user interface above the patient and provide inputs related to treatment. The arm is configured to place the display above the patient in relation to a probe inserted into the patient and view an image shown on the display, which can facilitate movement of the probes in response to the image shown on the display. The probe is located below the display, and the user is able to reach and adjust the probes while viewing images on the display. The user interface is configured with an image display area and a control panel, in which the control panel is located below the image display area, which allows the user to readily provide inputs to user interface while viewing the display and manipulating the one or more probes.

### RELATED APPLICATIONS

None.

### BACKGROUND

Prior methods and apparatus for treating patients can be less than ideal in at least some respects. Although surgical systems can be at least partially automated to perform at least some of the procedure in an automated manner and decrease user inputs, work in relation to the present disclosure suggests that the user interfaces of prior surgical systems can be less than ideal. For example, the display used to control the surgery may be less than ideally located in the operating room, which can result in the surgeon moving more often than would be ideal. Also, some of the controls of prior systems may be further from the surgeon than ideal, which can decrease the responsiveness of the surgeon to address the needs of the patient and may increase user fatigue. Although efforts to automate surgery have been successful, at least some of the prior systems can rely on dozens of user inputs to set up and plan a procedure, and the system controls may be less than ideally located, which may potentially result in less than ideal experience for the surgeon performing the surgery.

In light of the above, improved methods and apparatus are needed which would ameliorate at least some of the above mentioned aspects of prior surgical systems.

### SUMMARY

In some embodiments, user interface allows a user such as a surgeon to position a user interface comprising a display such as a touch screen display above the patient. In some embodiments, an arm is configured for a user to place the user interface above the patient, and provide inputs related to treatment. In some embodiments, the arm is configured to place the display above the patient in relation to one or more probes inserted into the patient and view one or more images shown on the display, which can facilitate movement of the one or more probes in response to the one or more images shown on the display. In some embodiments, the one or more probes are located below the display, and the user is able to reach and adjust the one or more probes while viewing images on the display. The user interface can be configured with an image display area and a control panel, in which the control panel is located below the image display area, which allows the user to readily provide inputs to user interface while viewing the display and manipulating the one or more probes.

In some embodiments, the display is configured to be placed above a midline of the patient, with one or more transverse images shown on the display approximately parallel to plane of the images, which can assist the surgeon with planning the treatment and moving the probes into alignment. In some embodiments the one or more probes comprises a longitudinal imaging probe configured to generate a longitudinal image such as a sagittal image, and the longitudinal image shown on the display is approximately parallel to the one or more images, which can assist the user such as a surgeon in understanding the physical location of the images in relation to the one or more probes.

### INCORPORATION BY REFERENCE

All patents, applications, and publications referred to and identified herein are hereby incorporated by reference in their entirety, and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIGS. 1A and 1B show a surgical system, in accordance with some embodiments of the present disclosure;

FIG. 2 shows alignment of a user interface, a treatment probe and an imaging probe of the system of FIGS. 1A and 1B, in accordance with some embodiments;

FIGS. 3A to 3F show a user interface comprising display, in accordance with some embodiments;

FIG. 4A shows a treatment probe configured for use with a surgical system, in accordance with some embodiments;

FIG. 4B shows a motor-pack configured to couple to a treatment probe, in accordance with some embodiments;

FIG. 5 shows treatment probe and corresponding movements of an energy source, in accordance with some embodiments;

FIG. 6A shows an arm configured to support a treatment probe, in accordance with some embodiments;

FIG. 6B shows mounting device configured to couple to a motor-pack in an unlocked configuration, in accordance with some embodiments;

FIG. 6C shows the mounting device of FIG. 6B in a locked configuration, in accordance with some embodiments;

FIG. 7 shows an ultrasound imaging device, in accordance with some embodiments;

FIG. 8 shows an arm configured to support the ultrasound imaging device of FIG. 7, in accordance with some embodiments;

FIGS. 9A and 9B show a front side view and a rear side view, respectively, of the surgical system of FIGS. 1 and 2 in a compact configuration, in accordance with some embodiments;

FIG. 10 shows side view of the surgical robotics system in a compact configuration, in accordance with some embodiments; and

FIG. 11 shows a method of treating a patient, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The presently disclosed systems and methods are well suited for use with many probes and diagnostic and surgical procedures. Although reference is made to a treatment probe comprising an energy source for prostate surgery and a transrectal ultrasound (“TRUS”) probe, the present disclosure is well suited for use with many types of probes inserted into many types of tissues, organs, cavities and lumens, such as brain, heart, lung, intestinal, eye, skin, kidney, liver, pancreas, stomach, uterus, ovaries, testicles, bladder, ear, nose, mouth, tumors, cancers, soft tissues such as bone marrow, adipose tissue, muscle, glandular and mucosal tissue, spinal and nerve tissue, cartilage, hard biological tissues such as teeth, bone, and lumens such as vascular lumens, nasal lumens and cavities, sinuses, colon, urethral lumens, gastric lumens, airways, esophageal lumens, trans esophageal, intestinal lumens, anal lumens, vaginal lumens, trans abdominal, abdominal cavities, throat, airways, lung passages, and surgery such as kidney surgery, ureter surgery, kidney stones, prostate surgery, tumor surgery, cancer surgery, brain surgery, heart surgery, eye surgery, conjunctival surgery, liver surgery, gall bladder surgery, bladder surgery, spinal surgery, orthopedic surgery, arthroscopic surgery, liposuction, colonoscopy, intubation, minimally invasive incisions, minimally invasive surgery, and others.

The presently disclosed systems and methods are well suited for combination with prior probes such as imaging probes and treatment probes. Examples of such probes include laser treatment probes, water jet probes, RF treatment probes, radiation therapy probes, ultrasound treatment probes, phaco emulsification probes, imaging probes, endoscopic probes, resectoscope probes, ultrasound imaging probes, A-scan ultrasound probes, B-scan ultrasound probes, 3D ultrasound probes, Doppler ultrasound probes, transrectal ultrasound probes, transvaginal ultrasound probes, longitudinal plane ultrasound imaging probes, sagittal plane ultrasound imaging probes, transverse plane ultrasound imaging probes, and transverse and longitudinal plane (e.g. sagittal plane) ultrasound imaging probes, for example.

The one or more images described herein can be generated in many ways, and may comprise one or more of a longitudinal image, a sagittal image, a parasagittal or a transverse image. In some embodiments, a longitudinal image comprises an image generated with an elongate imaging probe, in which the longitudinal image extends along a plane corresponding to an elongate axis of the imaging probe, and the transverse images extend along a plane that is transverse to the elongate axis of the probe, for example substantially perpendicular to the elongate axis of the probe and the corresponding longitudinal images. The elongate probe can be inserted into the patient with any suitable orientation. In some embodiments, the probe is inserted into the patient substantially along a midline of the patient, such that the longitudinal images correspond to sagittal images of the patient. In some embodiments, the elongate imaging probe comprises a TRUS probe, and the longitudinal images comprise sagittal images, although other probes with different orientations can be used to generate images in accordance with the present disclosure. Although reference is made to ultrasound probes inserted into the patient, in some embodiments the imaging device comprises an external imaging probe in which the longitudinal and transverse images can be referenced to one or more arrays of the external imaging probe. In some embodiments, the one or more images are generated from a 3D tomographic image data set such as a Digital Imaging and Communications in Medicine (DICOM) image data set.

The presently disclosed systems, methods and apparatuses are well suited for combination with many prior surgical procedures, such as water jet enucleation of the prostate, transurethral resection of the prostate (TURP), holmium laser enucleation of the prostate (HOLEP), prostate brachytherapy, and with surgical robotics systems and automated surgical procedures. The following patent applications describe examples of systems, methods, probes and procedures suitable for incorporation in accordance with the present disclosure: PCT/US2013/028441, filed Feb. 28, 2013, entitled “AUTOMATED IMAGE-GUIDED TISSUE RESECTION AND TREATMENT, published as WO 2013/130895; PCT/US2014/054412, filed Sep. 5, 2014, entitled “AUTOMATED IMAGE-GUIDED TISSUE RESECTION AND TREATMENT”, published as WO 2015/035249; PCT/US2015/048695, filed Sep. 5, 2015, entitled “PHYSICIAN CONTROLLED TISSUE RESECTION INTEGRATED WITH TREATMENT MAPPING OF TARGET ORGAN IMAGES”, published as WO2016037137; PCT/US2019/038574, filed Jun. 21, 2019, entitled “ARTIFICIAL INTELLIGENCE FOR ROBOTIC SURGERY”, published as WO2019246580A1 on Dec. 26, 2019; PCT/US2020/021756, filed Mar. 9, 2020, entitled “ROBOTIC ARMS AND METHODS FOR TISSUE RESECTION AND IMAGING”, published as WO/2020/181290; PCT/US2020/058884, filed on Nov. 4, 2020, entitled “SURGICAL PROBES FOR TISSUE RESECTION WITH ROBOTIC ARMS”, published as WO/2021/096741; PCT/US2021/070760, filed on Jun. 23, 2021, entitled “INTEGRATION OF ROBOTIC ARMS WITH SURGICAL PROBES”, published as WO/2021/263276; and PCT/US2021/038175, filed on Jun. 21, 2021, entitled “SYSTEMS AND METHODS FOR DEFINING AND MODIFYING RANGE OF MOTION OF PROBE USED IN PATIENT TREATMENT”, published as WO/2021/262565; the entire disclosures of which are incorporated herein by reference.

In some embodiments, an energy source is used to treat tissue. The energy source may comprise any suitable energy source, such as one or more of an electrode, a loop electrode, a laser source, a thermal energy source, a mechanical energy source, a mechanical sheer, an ultrasound probe, a cavitating ultrasound probe, a water jet, e.g. a fixed pressure water jet, a plasma source, a steam source, a morcellator, a trans urethral needle, a photo ablation source, a radiation energy source, a microwave energy source or a water jet evacuation source. The energy source can be combined with other treatments and compounds, such as photochemical treatment agents. The imaging probe may comprise any suitable probe, such as endoscopic probes, resectoscope probes, ultrasound imaging probes, A-scan ultrasound probes, B-scan ultrasound probes, Doppler ultrasound probes, transrectal ultrasound probes, transvaginal ultrasound probes, sagittal plane ultrasound imaging probes, transverse plane ultrasound imaging probes, and transverse and sagittal plane ultrasound imaging probes, for example.

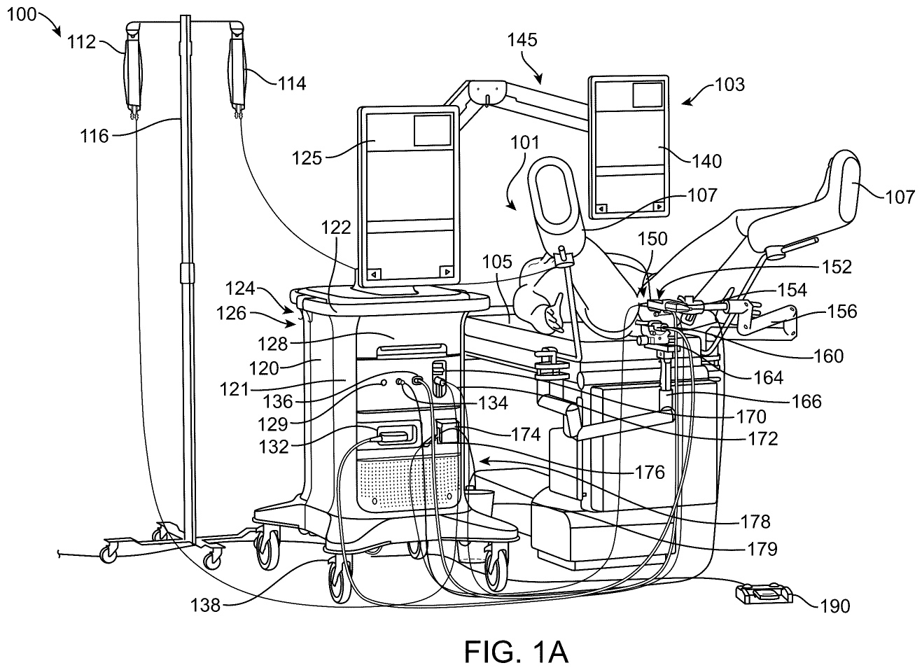

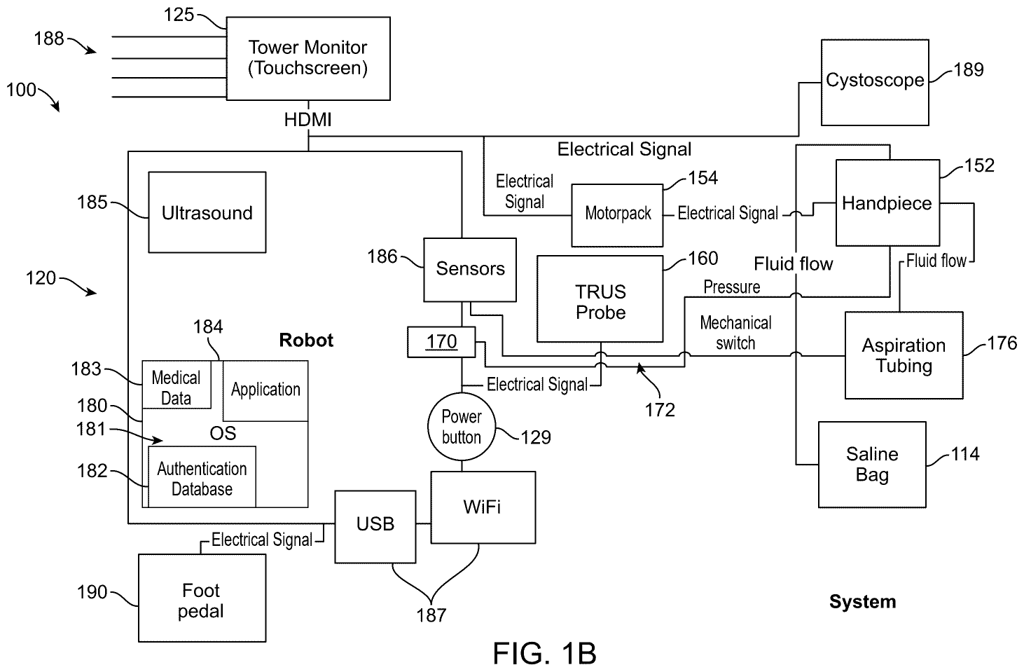

FIG. 1A shows a surgical system 100, in accordance with some embodiments. The system 100 comprises a console 120 which comprises several components of the system 100, such as electronics, circuitry and one or more processors to control a treatment probe 150, which has been inserted into the patient 101. The system 100 comprises a user interface 103 comprising a monitor, such as a touch screen display 140. The display 140 is coupled to an adjustable arm 145, which allows the display to be positioned above a midline of patient near an insertion location of the probe 150, which can allow the user such as a surgeon to control the surgery when seated in front of the display 140 and near the surgical instruments, such as the treatment probe 150 and an imaging probe 160.

The console 120 can be configured and sized and shaped in many ways. In some embodiments, the console 120 comprises a tower, in which the horizonal dimensions of the console are less than a vertical height of the console. Alternatively or in combination, the console 120 may comprise a cart, for example with lockable wheels, which allow the console to be moved.

The user interface 103 can be configured in many ways and may comprise a display, such as a touch screen display, for example. Although reference is made to a touch screen display, the user interface 103 positioned above the patient may comprise one or more components that are not shown on the display, such as a positioning pad, one or more input buttons, a pointing device such as a track pad, a knob, or other suitable component. These components of the user interface 103 can be located to the side of the display 140, for example. Alternatively or in combination, the display may comprise one or more input features shown on the touch screen display touch screen display, such as buttons, a positioning pad, and features configured for the user to swipe the display to provide user input.

In some embodiments, the system 100 comprises a patient support 101. The patient support may comprise any suitable support, such as a bed, for example. The support 101 can be configured to support the patient such that a midline of the patient is approximately aligned with a midline of the support 101. The support 105 may comprise one or more stirrups 107, which allow the legs and feet of the patient to be elevated during surgery. In some embodiments, the support 101 is configured to place the patient in a lithotomy position, for example. The support may comprise adjustable features such as one or more motors to raise and lower the patient for example.

In some embodiments, the system 100 comprises one or more probes inserted into the patient. The one or more probes may comprise a treatment probe 150. While the treatment probe 150 can be configured in many ways, in some embodiments the treatment probe 150 comprises an energy source configured to deliver energy to the patient to treat tissue as described herein. In some embodiments, the treatment probe is configured to be inserted into an external opening of the urethra of the patient, although other access locations can be used, such as surgical opening to introduce the probe. In some embodiments, the one or more probe inserted into the patient comprises an imaging probe, such as an ultrasound probe, for example. In some embodiments, the imaging probe inserted into the patient comprises a transrectal ultrasound (TRUS) probe 160. Although reference is made to a separate ultrasound probe, in some embodiments the ultrasound imaging transducer array is inserted into the patient through the same opening as the treatment probe and may located on the same probe as the energy source. Also, the ultrasound imaging device may comprise an external ultrasound imaging array, for example.

The one or more probes inserted into the patient may be coupled to one or more arms to support the one or more probes. The one or more arms may comprise a treatment probe arm 156 configured to support the treatment probe 150 during treatment, for example with the treatment probe 150 inserted into the patient. Alternatively or in combination, the one or more arms may comprise an arm to support the imaging probe, such as an arm 166 configured to support a TRUS imaging probe. In some embodiments, the TRUS probe is coupled to the arm 166 with a stepper 164, which allows the probe 166 to be advanced along the rectum or the colon of the patient and to be drawn proximally toward the surgeon with rotation of a knob with the TRUS probe 160 inserted into the patient 101. In some embodiments, the stepper 164 comprises a cradle for rotationally adjusting the TRUS probe 160 to align with patient anatomy.

The treatment probe 150 can be configured in many ways, and may comprise a handpiece 152 configured to allow the probe to be inserted into the patient. The handpiece 152 can be configured to couple to a source of mechanical energy, such as a motor-pack 154, which is configured to drive one or more components of the treatment probe. In some embodiments, the treatment probe is configured to receive an endoscope such as a cystoscope in order to allow visualization of the treatment area and to facilitate insertion of the treatment probe. In some embodiments, the endoscope is incorporated into the probe as an integral part of the probe.

The console 120 can be configured in many ways, and may comprise internal electronics, processors, circuitry and connectors configured to couple to other components of the surgical system. In some embodiments, the console 120 comprises one or more handles 122 and wheels such as locking wheel 138, which allow the console to be moved by up to two people at a time, for example to be stored or brought into an operating room for surgery and locked into position. The console 120 may comprise additional features to facilitate surgery and use of the system, such as one or more of a motor-pack holder 124, a TRUS probe holder 126, a drawer 128, or a power button 129, for example. In some embodiments, the motor-pack holder 124 and the TRUS probe holder can located on either side of the console 120, depending on user needs. The console 120 may comprise connectors to couple to one or more components of the surgical system 100, such as one or more of a TRUS probe connection 132 configured to couple the console to the TRUS probe 160, a motor-pack connection 134 configured to couple to the motor-pack 154, or a scope connection 136 configured to couple to an endoscope such as a cystoscope.

In some embodiments, the system 100 comprises a water jet pump cartridge 170, which is received in a receptacle of console 120. In some embodiments, a water jet line 172 extends from the pump cartridge 170 to the treatment probe 150 to provide a water jet from the treatment probe. In some embodiments, the console 120 comprises an aspiration pump 174, which is coupled to probe 150 with an aspiration line 174.

In some embodiments, the system 100 comprises a waste line 178 configured to couple to a waste container 179. The waste line 178 can be configured to receive fluids and other material from the patient via the treatment probe 150, for example. In some embodiments the waste line 178 is coupled to the aspiration pump 176, although line 178 may receive fluids from other sources, for example.

In some embodiments, the system 100 comprises a foot pedal 190 configured for a user such as a physician to control treatment, such as starting, pausing, resuming or stopping the treatment for example.

In some embodiments, the console 120 comprises a front side 121 in which a plurality of connectors are plugged into the console 120, which allows the user such as a surgeon to readily view the connectors to ensure that these and the associated lines have been properly connected. The plurality of connectors located on the front side 121 may comprise one or more of the TRUS probe connection 132, the motor-pack connection 134, the scope connection 136, the water jet pump cartridge 170, the water jet line 172, the aspiration pump 174, the waste line 178, or the connector of the foot pedal 190. This location of the connectors and lines allows a user such as a surgeon to readily view the connectors to ensure that the appropriate connections have been made, and may help diagnose system 100 if a fault is detected, for example.

In some embodiments, system 100 comprises a source of fluids, such as a liquid, e.g. saline, although other fluids such as gasses for insufflation may be provided. In some embodiments, a saline source 112 is coupled to water jet pump cartridge 170. In some embodiments, a saline source 114 is coupled to treatment probe 150 in order to flush the surgical site with a fluid such as saline, for example. In some embodiments, one or more of the saline sources is supported with a pole 116 to provide saline to one or more of the pump or the probe 150 with gravity assisted flow, for example. The pole 116 may comprises a pole that is separate from the console 120 or a pole that is integrated with the console 120, for example so as to extend upwardly from the console.

In some embodiments, the system 100 comprises a second display 125, which is supported with the console 120. The second display 125 can be helpful for staff to view the surgery and to assist the surgeon. In some embodiments, the second display 125 mirrors display 140. The second display 125 may have similar user input features as the display 140, or decreased functionality to ensure the physician retains control of the treatment. In some embodiments, the screen of the second display 125 comprises annotation features for staff or a proctor to provide annotation inputs for the surgeon to view on the first display 140, for example without controlling system functions from the second display.

FIG. 1B shows a schematic diagram of some components of system 100. In some embodiments, the console 120 comprises one or more processors 180. The one or more processors 180 can be configured with instructions to perform various functions of system 100. In some embodiments, the one or more processors 180 comprise an operating system 181, an authentication database 182 to provide user authentication, a medical database to store medical data of patients, an application block 184 to run one or more applications of the system, such as treatment planning and user interface (UI) processes, instructions to the motors to move the linkage of treatment probe 150, and system monitoring functions as describe herein. In some embodiments, the console 120 comprises imaging circuitry such as ultrasound circuitry 185 housed within the console 120. In some embodiments, the one or more processors 180 is configured to give different users different levels of access 188 such as full system access or partial access to system 100. The access 188 can be provided through a console user interface, for example via the console display 125, which may comprise a touch screen display, for example. In some embodiments, the system access is provided to administrators, such as a urology administrator of the local health care provider, hospital information technology administrators, and representatives of the manufacturer of system 100, such as field service representatives. In some embodiments, the system 100 comprises communication circuitry 187 housed within console 120, such as universal serial bus (USB) circuity and wireless fidelity (WiFi) circuitry, for example.

While the console 120 can be configured in many ways, in some embodiments, the console 120 comprises internal components, such as an internal computer system and an internal ultrasound imaging system housed within console 120. In some embodiments, the console 120 comprises a tower configuration, in which the console comprises a height greater than a width of the console.

The console 120 can be coupled to the components of system 100 with one or more connectors such as electrical connectors or fluidic connectors as described herein. In some embodiments, the console 120 is coupled to the console display 125 with an electrical connector such as an HDMI cable. The console can be connected to the endoscope such as cystoscope 189 with an electrical line to transmit electrical signals, in order to display images on the displays. The motor-pack 124 can be coupled to the console 120 with one or more electrical lines such as cables, to transmit movement signals and provide feedback to the one or more processors within console 120. The motor-pack 154 can be coupled to the handpiece 152 with one or more electrical lines to transmit electrical signals between the handpiece and the motor-pack 154 and the one or more processors 180, such as encoder signals and feedback signal related to movement of the energy source on the probe provided by a linkage housed within the handpiece 152. The one or more sensors 186 can be coupled to the one or more processors 180 with internal circuitry housed within console 120 to monitor fluid flow, for example. The one or more sensors 186 can be coupled to the aspiration pump, for example. In some embodiments, the one or more sensors 186 is coupled to the handpiece 152 with aspiration tubing 176 to monitor the removal of material from handpiece 152. In some embodiments, the saline source 114 such as the saline bag is coupled to the handpiece 152.

In some embodiments, the ultrasound imaging device such as TRUS probe 160 is coupled to the console 120 with one or more electrical lines, such as an electrical cable to couple the ultrasound imaging device such as TRUS probe 150 to the ultrasound circuitry 185.

In some embodiments, the console 120 comprises one or more sensors 186 coupled to the aspiration line 176 to measure fluid flow from the handpiece 152.

In some embodiments, the system 100 comprises an endoscope such as cystoscope 189, configured to view optical images of tissue at the surgical site. The endoscope such as cystoscope 189 may be coupled to the handpiece 152 and the probe 150 as described herein.

In some embodiments, a pump cartridge 170 is insertable into a receptacle of the console, in order to provide pressurized water to the energy source such as a waterjet on the treatment probe. In some embodiments, the high pressure line 172 couples the pump cartridge to the water jet on the handpiece.

In some embodiments, the foot pedal 190 is coupled to the console 120 with one or more electrical lines to transmit signals, such as a cable, in order to control system 100 as described herein.

The arm 145 can be configured in any way to support the surgeon display 140 above the patient as described herein. In some embodiments, the arm 145 comprises a plurality of joints and one or more of a locking arm, a clutch arm, a manual arm, a gas-spring arm, a robotic arm, a zero-gravity arm or a zero gravity robotic arm, for example. In some embodiments, the arm 145 comprises a gas spring arm with a gas strut and a spring.

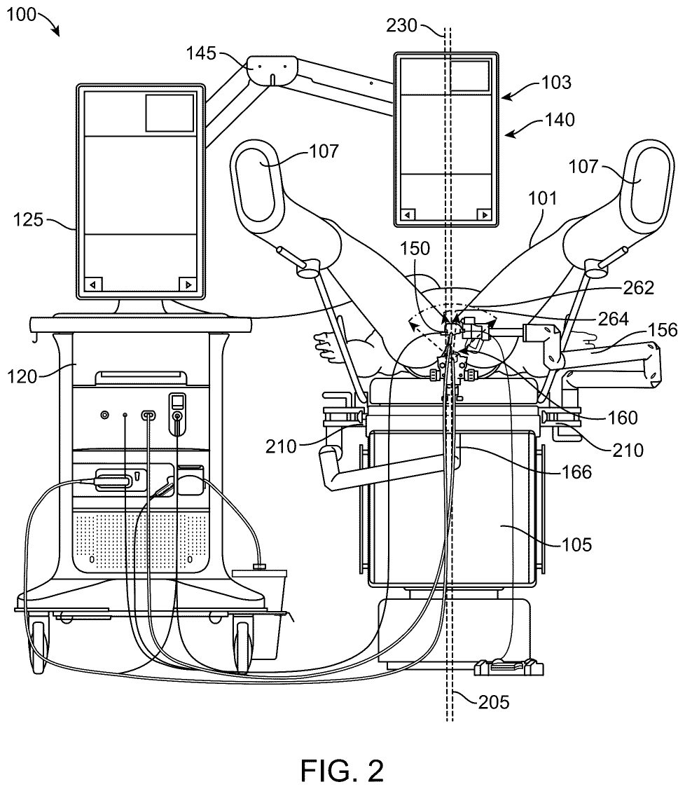

FIG. 2 shows alignment of a user interface 103, a treatment probe 150 and an imaging probe 160 of the system 100, in accordance with some embodiments. In some embodiments, the patient comprises a midline 205, and the user interface 103 and the treatment probe 150 are arranged with respect to the midline of the patient. In some embodiments, the monitor is poisoned such that a plane corresponding to the midline of the patient extends through the monitor, which can help with visualization of the images shown on the display. This location is also helpful because it allows the surgeon to readily reach the input controls of the user interface 103, which comprises the display 140. Readily reaching the input controls can be helpful to decrease fatigue associated with controlling the surgical system 100, for example when several surgeries are performed in a day or with long surgeries lasting more than an hour, for example.

In some embodiments, the arm 145 is configured to support the user interface 103 comprising the surgeon display 140 coupled to the positioning arm, in which the positioning arm 145 is configured to support the display at a first location above a midline of the patient with a first configuration. In some embodiments, the arm 145 is configured to place the surgery display 140 at a second location away from the midline of the patient with a second configuration for storage as described herein.

While the arm 145 can be configured in many ways, in some embodiments the arm is configured to allow movement of the surgeon display 140 for visualization and interaction by the surgeon while surgeon may be on one knee for insertion of the ultrasound probe such as TRUS probe 160, for example. In some embodiments, the display is configured to receive user inputs when covered with a sterile drape, and the inputs are provided through the sterile drape, and the sterile drape may comprise a substantially transparent drape to allow the user to see the display through the drape and provide inputs through the drape. The arm 145 can be configured for the surgeon to move the display 140 out of the way during handpiece insertion, for example by pushing on display 140 to rotate the arm 145 relative to the console 120 to position the display away from the surgeon and superiorly above the patient, for example while using sterile technique with display at least partially draped and the surgeon wearing surgical gloves. The display 140 can then pulled in toward the physician while seated or standing for touch-screen intensive steps after insertion and alignment of probes as described herein.

In some embodiments, the imaging probe such as TRUS probe 150 is arranged with respect to the midline of the patient, the treatment probe 150 and the display 140. In some embodiments, this arrangement can assist the physician with understanding the physical locations of the probes in front of them and coordinating movements of the probes in relation to internal images of the patient shown on the display. For example, the physician can move a proximal end of a probe in order to manually move a distal portion of the probe, e.g. manually translate or rotate the probe, or use instrument controls from the user interface 103 in order to move one or more of the probes to a desired location within the patient, such as with translation or rotation of the probe.

In some embodiments, the imaging device such as TRUS probe 160 is configured to generate longitudinal images such as sagittal images and transverse images. In some embodiments, the longitudinal images correspond to an imaging plane along with a longitudinal image of the probe, and the transverse images correspond to images along plane transverse to the elongate axis of the imaging probe. In some embodiments, the transverse images correspond to a transverse field of view 262, which corresponds to a transverse image plane. In some embodiments, the transverse field of view corresponds to an angle of rotation about an elongate axis of the imaging probe such as TRUS probe 260. With the monitor placed over the midline of the patient, the physician can more readily associate the transverse images shown on the display with the physical locations of the corresponding tissue that is shown in the images.

In some embodiments, the imaging device such as TRUS probe 260 is configured to generate longitudinal images, e.g. sagittal images, along a longitudinal image plane within a longitudinal image field of view 624, which may comprise a sagittal image along a sagittal image field of view. In some embodiments, the monitor is aligned with the patient at a location above the sagittal image plane, such that the plane of the sagittal image extends through the monitor, for example. This alignment of the display with the longitudinal image plane allows the physician to associate the position of the probes more readily with the longitudinal images. Alternatively or in combination, the monitor can be located inferiorly or superiorly, with respect to the patient, such that the plane of the transverse image is within about 10 inches of the image display area of the monitor. In some embodiments, this positioning of the monitor can improve the physician's ability to associate the transverse and sagittal images shown on the display with the physical locations of the probes and corresponding tissue that is shown in the images.

In some embodiments, the transverse image shown on the display approximately parallel to the transverse image plane. In some embodiments, the transverse image plane is parallel to the image shown on the display to within 30 degrees, or within 10 degrees, for example. In some embodiments, the ultrasound imaging device comprises an ultrasound array configured to generate a longitudinal image along a longitudinal image plane, the arm configured to support the display with longitudinal image displayed on the display above the patient.

In some embodiments, the longitudinal image plane corresponds to a plane extending from the ultrasound transducer array and through a portion of the display. In some embodiments, the transducer device is rotatable about a longitudinal axis in order to rotate an angle of the longitudinal image plane to view the treatment probe with the plane extending through the transducer array, the treatment probe and the monitor. In some embodiments, the treatment probe extends through at least a portion of the longitudinal image plane. In some embodiments, the longitudinal image shown on the display is approximately perpendicular to the longitudinal image plane and optionally to within 30 degrees of perpendicular.

In some embodiments, the ultrasound imaging device such as TRUS probe 160 comprises a transverse array and a longitudinal array as described herein, and the arm 145 is configured to position the image shown on the display to within 30 degrees of parallel to a long axis of the longitudinal array and to within 30 degrees of perpendicular to a long axis of the transverse array.

In some embodiments, patient support 105 is configured to support the patient with the patient support below patient, the midline 205 of the patient corresponding to a plane extending through the display and the support.

In some embodiments, the stirrups 107 are configured to support the legs of the patient splayed and the feet of the patient above a torso of the patient, and wherein the surgeon display 140 is sized to fit between the legs of the patient.

In some embodiments, the arm 145 is configured to support the surgeon display 140 above the patient and superiorly relative to a penile fenestration of a drape covering the patient, which the treatment probe 150 enters the patient.

While the surgeon user interface 103 and display 140 can be configured in many ways, in some embodiments, display 140 has a height and a width, wherein the height is greater than the width. In some embodiments, the positioning arm 145 is configured to align a vertical centerline 230 of the display 140 with a midline of the patient 205 and a corresponding midline of the patient support. In some embodiments, the positioning arm 145 is configured to place a vertical centerline 230 of the display within about 2 inches of a midline 205 of the patient with the patient on the support 105.

In some embodiments, the positioning arm 145 is configured to place a centerline of the touchscreen display 140 to within 2 inches of a centerline of the treatment probe, for example a longitudinal axis of probe 150.

In some embodiments, the surgeon display 140 comprises a touch screen display configured to receive a plurality of user inputs through a drape placed over the touch screen display to provide sterility. The arm 145 is configured to resist movement in the extended configuration in response to the user input through the drape. In some embodiments, the touchscreen display 140 is configured to receive a plurality of user inputs from a finger of a user covered with a glove transmitted from the finger through the glove and the drape to the touchscreen display, and the arm is configured to resist movement in response to the user input transmitted through the glove and the drape. In some embodiments, the drape comprises a sterile drape and the glove comprises a sterile glove.

While the arm 145 can be configured in many ways, in some embodiments the arm comprises a locking arm configured to lock the arm in place in the extended configuration above the patient. In some embodiments, movement of the display is inhibited with a locking mechanism, such as a turn screw, lever or clamp, for example.

Alternatively or in combination, the arm can be configured not to move substantially when subjected to forces associated with inputs to display 140, such as touch inputs, and to move in response to greater amounts of force to reposition the display 140 supported with arm 145. In some embodiments, the positioning arm is configured to resist movement of the touchscreen display when placed above the midline 205 and touched by a user with a first amount of force to input data and to allow movement of the display with a second mount of force greater than the first amount of force. In some embodiment, the first amount force is within a range from about 0 pounds (e.g. 0.01 or 0.1 pounds) to about 5 pounds and optionally within a range from about 0 (e.g. 0.01 or 0.1) pounds to about 2 pounds. In some embodiments, the second amount of force is greater than about 2 pounds and optionally greater than about 5 pounds. The arm can be configured with stiction to inhibit movement of the display when the first amount of force is applied touch screen when the touch screen is positioned above the midline of the patient and to allow movement with the second amount of force greater than the first amount of force, for example. The arm 145 may comprise frictional pads configured to resist movement with forces below the threshold amount of force and to allow movement with forces above the threshold amount.

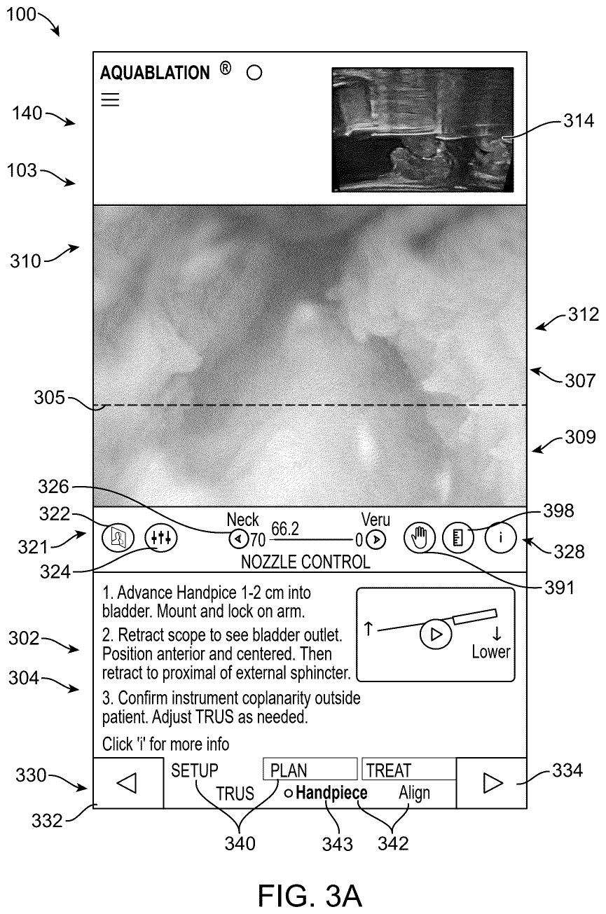

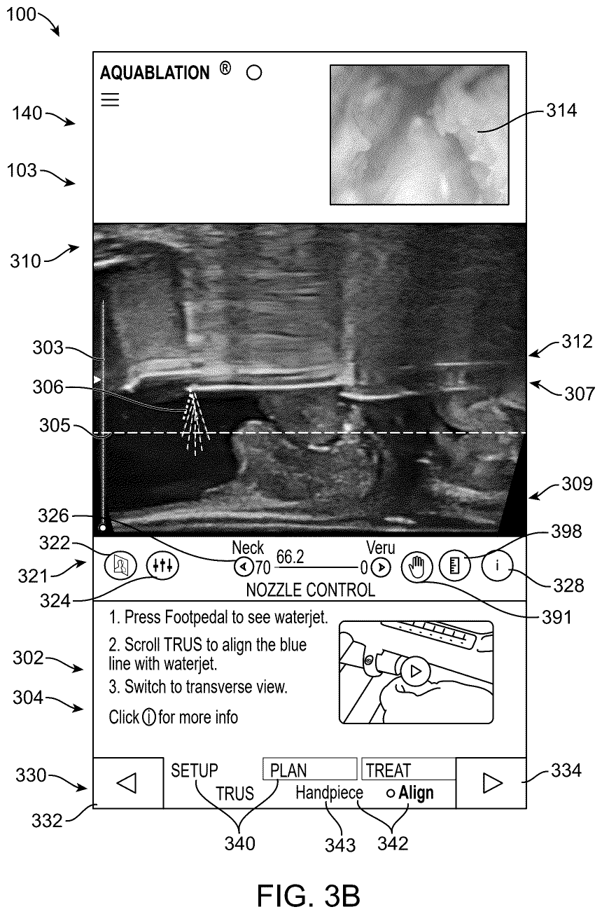

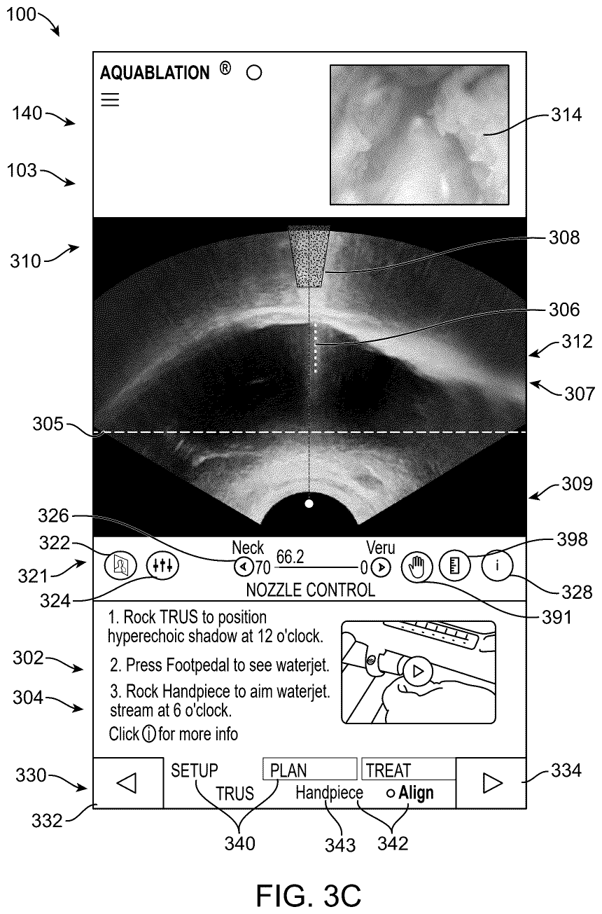

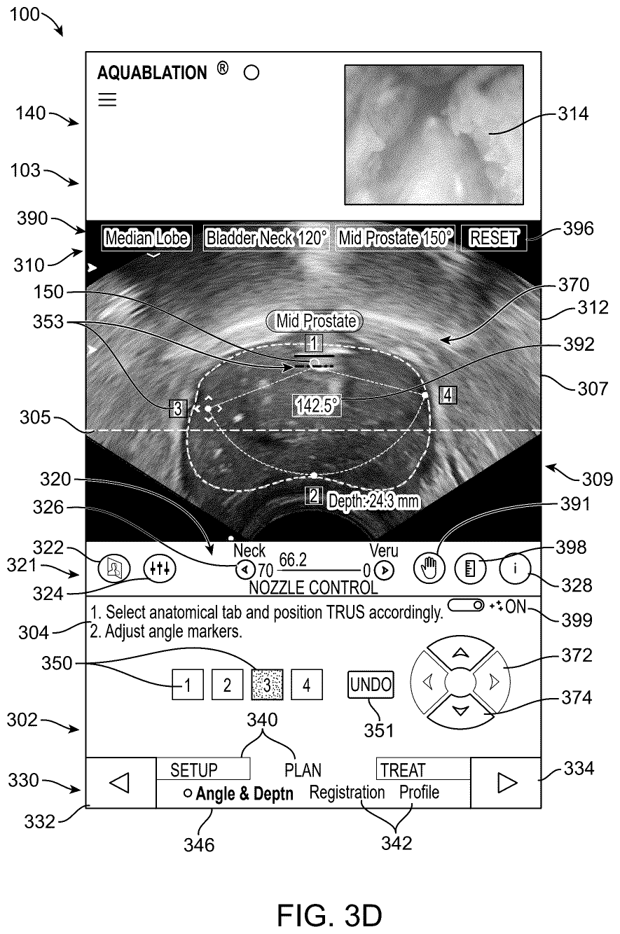

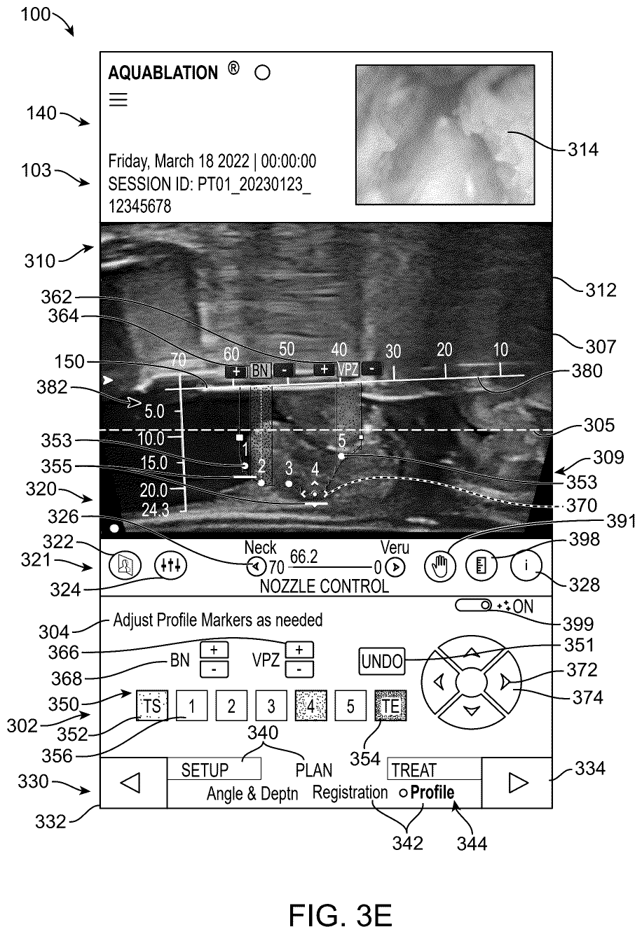

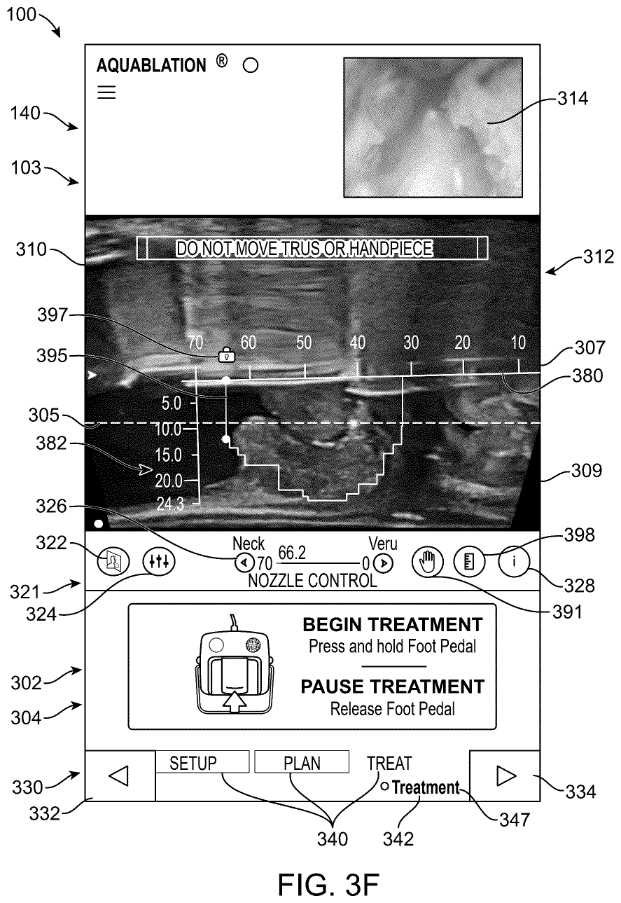

FIGS. 3A to 3F show a user interface 103 comprising a display 140 of the system 100, in accordance with some embodiments. In some embodiments, the display 140 comprises a portrait orientation to improve placement of the monitor above the patient as described herein. In some embodiments, the user interface 103 comprises an image display area 310 and a control panel 302, in which the control panel 302 is partitioned from the image display area. In some embodiments, the control panel 302 is located on an area of the display that is separate from the image display area 310, such that the control panel and image display area appear on different areas of the display. In some embodiments, the control panel 302 is located below the image display area 310. Alternatively, the control panel can be located above the image display area or alongside the image display area, for example with the display in a landscape configuration.

Although reference is made to a control panel 302 in the context of a touch screen display, one of ordinary skill in art will recognize that the control panel 302 may comprise one or more hardware components instead of a touchscreen area of the display 140 as described herein. The control panel 302 located below the image control area can facilitate user control and decrease the likelihood that the user will reach as far upward to provide inputs to user interface 103 as compared to the image control panel being located above the image display area.

In some embodiments, the user interface 103 is configured for the user to touch the user interface such as the touchscreen display a total number of times at a plurality of locations to set up the treatment, plan the treatment and perform the treatment, and at least half of the plurality of locations touched the total number of times is located below a centerline of the display, which can improve ergonomics and facilitate the user providing input to the user interface. In some embodiments, at 75% of the plurality of locations touched the total number of times is located below a centerline of the display. In some embodiments, at 90% of the plurality of locations touched the total number of times is located below a centerline of the display. In some embodiments, the total number of times the user touches the user interface at the plurality of locations to set up the treatment, plan the treatment and perform the treatment comprises a total number of touches at least 50 times, a total number of touches of at least 100 times, a total number of touches of at least 150 times, a total number of touches of at least 200 times, or a total of number of touches of at least 250 times.

In some embodiments an upper area 307 of the display is located above a horizontal centerline 305 and a lower area 309 of the display is located below the horizontal centerline 305. In some embodiments, at half of the image display area 310 is located above the centerline and at least half of the area of the control panel 302 is located below the centerline 305, which can improve ergonomics because the control panel can be more readily reached by the user as compared to the control panel located above the image display area. In some embodiments the user will touch the control panel at least twice as often as the image display area, and in some embodiments the user will touch the control panel at least four times as often as the image display area. In some embodiments, at least 75% of the image display area 310 is located above the horizontal centerline and at least 75% of the control panel is located above the image display area. In some embodiments, at least 90% of the image display area 310 is located above the horizontal centerline and at least 90% of the control panel is located above the image display area. In some embodiments, the entire image display area 310 is located above the horizontal centerline and the entire control panel is located above the image display area.

The image display area 310 can be configured in many ways and may comprise a primary image area 312 and a secondary image area 314. The secondary image display area can be located outside the primary image display area. Alternatively or in combination, the secondary image may be shown within a portion of the primary image, for example with a picture in picture configuration. In some embodiments, the primary image display area is larger than the secondary image display area.

The control panel 302 can be configured in many ways, and may comprise a tool bar 321, for example. The tool bar 302 may comprise one or more of a plane toggle icon 302 for a user to toggle between sources of the primary image and the secondary image in response to a user input to the icon, an image setting icon 324 for a user to select to adjust image settings, a longitudinal control icon 326 for a user to adjust a longitudinal position of an energy source on the treatment probe, a measurement icon 398 for a user to select to measure distances between structures shown in an image, or an information icon 328 for a user to select to display information such as information about the surgical system and the patient, for example. Although reference is made to plane toggle icon 302, image setting icon 304, longitudinal control icon 326, measurement icon 398 and information icon 328 in the context of tool bar 321, one or more of these icons and controls can be located elsewhere on the display, for example.

The icons shown on the display may comprise any suitable symbol to indicate the functionality of the icon. In some embodiments, the icon is configured for a select the icon with a user input such as touching the display at a location corresponding to the icon. In some embodiments, the plane toggle icon 322 is configured to receive a user input such as the user touching the icon to toggle the primary image and the secondary image source.

The settings icon such as the image setting icon 324 can be configured in many ways, and may comprises any suitable icon such as a gear, slider or other icon that conveys to the user that settings can be adjusted with the icon. In some embodiments, the image setting icon 324 shows a plurality of sliders to indicate to the user that the user can adjust the image settings. In some embodiments, the user touching an icon to provide user input results in a pop up window, such as a pop up window for the user to adjust the imaging settings, which may comprise imaging setting of one or more of the endoscope or the TRUS probe. In some embodiments, the icon may comprise a plurality of controls associated with the icon. For example, the longitudinal control icon 326 may comprise a first icon such as an arrow pointing in a first direction to indicate to the user that an energy source is moved in the first direction in an image shown on the display, e.g. to advance a probe carrying the energy source, and a second icon such as an arrow pointing in a second direction to indicate to the user that the energy source is moved in the second direction in an image shown on the display, e.g. to retract the probe carrying the energy source. The longitudinal control 326 may comprise a third icon, such as a bar, to show the position of the energy source on the probe, for example.

In some embodiments, the user interface is configured to work with a first display 140 such as a surgeon display and a second display 125 for staff and other people associated with the surgical procedure. In some embodiments, a display control icon 391 allows a user to adjust input settings of the first display 140 and the second display 125 to allow limited inputs with the second display 125 while the surgeon controls the inputs with surgeon display 140. In some embodiments, the display control icon 391 comprises an annotation enable toggle that allows annotations to be input from the second display 125 when enabled and does not allow annotations to be input from the second display 125 when disabled. In some embodiments, the display control icon 391 is configured to provide a pop-up window that allows the user of the first display 140 to allow at least some user input from the second display 140, such as annotations. In some embodiments, the first display 140 comprises a touch screen display configured for a user such as a surgeon to control and plan the surgery with inputs as described herein, and the second display 125 is configured to substantially mirror the first display 140, which allows the operating room staff to readily view the surgical procedure on the second display 125 in synchrony with the surgeon viewing the first display 140.

In some embodiments, only the first display 140 is configured to accept and confirm the treatment plan from the surgeon touchscreen display 140, even when annotations at the second display are enabled. In some embodiments, the second display 125 is configured to allow limited user inputs such as annotations without adjusting treatment parameters or accepting the treatment plan, and the annotations are mirrored on both the first display 140 and the second display 125, such that the annotations can be viewed on both displays without allowing control of the surgical procedure or surgical input parameters from the second display 125. The annotations provided with input to the second display 125 can be helpful to communicate with the surgeon and can be used for telestration and tele-proctoring, for example. Although reference is made to a first display and a second display, additional displays can be provided. For example a remote display can be configured to allow annotations that are viewed on displays 125 and 140, which can allow remote telestration and remote proctoring, for example. In some embodiments, the second display 125 comprises a remote display, such as a display in another room, another building, another state, or another country, for example.

In some embodiments, the control panel 302 provides one or more instructions 304 to the user. The instructions may comprise any suitable instruction, such as an instructions to be perform at next step at a current stage in the procedure, or an instruction to view a video, for example.

In some embodiments, the control panel comprises a navigation bar 330. The navigation bar may comprise icons for the user to navigate through stages of the procedure. In some embodiments, the navigation bar 330 comprises a previous icon for the user to go back to a prior screen and step of the procedure, and a next icon for the user to advance to a next screen and step of the procedure.

In some embodiments, the navigation bar 330 comprises one or more indicators for a user to assess progress of the navigation through different stages of the procedure. In some embodiments, the user navigation bar 330 is configured to display a plurality of modes 340 of the system 100, such as one or more of a set up mode, a plan mode or a treatment mode. Each of the modes may comprise a plurality of sub-modes, in which each of the corresponding sub-modes corresponds to a stage of the associated mode. In some embodiments, the setup mode comprises one or more of a TRUS sub-mode to insert the ultrasound probe such as a TRUS probe, a handpiece mode for the user to insert the treatment mode with the handpiece, and an align mode for the user to align the treatment probe and the TRUS probe with each other to visualize the treatment probe with the TRUS probe. In some embodiments, the plan mode comprises an angle and depth sub-mode for the user to determine a plurality of angles and depths of treatment from the probe on a plurality of transverse images, a registration sub-mode for the user to register the position of the energy source along a longitudinal image such as a sagittal image, and a profile sub-mode for the user to adjust markers associated with a positions of the treatment plan on the longitudinal image such as a sagittal image.

Generally, an icon of the user interface 103 is highlighted it appears differently relative to other icons, for example if its contrast, boldness, color or shape on the display relative to other icons is different than the contrast, boldness, color or shape of other, non-highlighted icons. In some embodiments, a highlighted icon comprises an icon that is more visible than non-highlighted icons.

Referring to FIG. 3A, the set up mode is highlighted relative to other modes of the plurality of modes 340, such as relative to the plan mode icon and the treat mode icon. The handpiece sub-mode 343 is highlighted relative to the other sub-modes of the plurality of sub-modes 342, such as the TRUS sub-mode and the Align sub-mode.

Although reference is made to the plurality of modes and corresponding sub-modes in the context of a navigation bar, these modes and corresponding sub-modes can be presented on the display outside the navigation bar.

In some embodiments, the UI 103 is configured for the user to press the next icon 334 to proceed to the next stage of the procedure, although the user may press the previous icon 332 to proceed to a prior stage of the procedure.

Referring to FIGS. 3B and 3C, the UI 103 is configured to align the treatment probe 150 and ultrasound device such as TRUS probe 160.

As shown in FIG. 3B, the primary image 312 comprises a longitudinal ultrasound image such as sagittal image from TRUS probe 150, and the secondary image 314 comprises the endoscope image such as cystoscope image. The one or more modes 340 comprises the setup mode and the one or more sub-modes 342 comprises the align mode with the align sub-mode highlighted in relation to the other sub-modes 342, such as the hand piece mode 343. One or more markers 303 shows a position of the display corresponding to an end of the ultrasound probe such as TRUS probe 160. In some embodiments, the path of the energy 306 from the energy source is visible on the primary image 312 shown on the display.

In some embodiments, the processor is configured with instructions to automatically change the source of the primary image 312 shown on the display in accordance with the current task to be performed by the user, for example a task associated with the current mode of the one or more modes 340 and the current sub-mode of one or more sub-modes 342. As shown in FIG. 3B, the UI has changed the primary image 312 to a real time ultrasound image and the secondary image 314 to the real time endoscope image. In some embodiments, the user interface is configured to change the primary image 312 from the real time endoscopic image such as the cystoscope image to the real time ultrasound image, for example to a longitudinal image such as the sagittal image in response to the stage of treatment progressing from the handpiece sub-mode 343 to the alignment sub-mode. In some embodiments, the processor is configured to automatically change the primary image 312 of the user interface 103 in response to the user progressing to another stage of a mode or sub-mode, such as changing the primary image from a longitudinal image such as a sagittal image, for example. In some embodiments, the secondary image 314 comprises the real time endoscope image such as the cystoscope image while the user interface or the user changes the ultrasound image shown as the primary image 312.

In some embodiments, the one or more user instructions 304 are configured to instruct the user. The one or more user instructions 304 may comprise a single displayed instruction or a plurality of instructions, for example. In some embodiments, the user is instructed to press on the foot pedal to release energy from the energy source, in order to detect the position of the energy source such as a water jet, although other energy sources can be detected on the primary image as described herein. In some embodiments, the user is instructed to translate the imaging device such as the TRUS probe along a longitudinal axis of the imaging probe to align the one or more markers 303 with the energy source such as the water jet. In some embodiments, the user is instructed to provide an input such as a click or touch on the information icon 328 to obtain additional information about the current mode and sub-mode, for example. In some embodiments, the user is instructed to change the view from the imaging device from a longitudinal mode such as a sagittal mode to a transverse mode.

In some embodiments, the UI 103 is configured for the user to press the next icon 334 to proceed to the next stage of the procedure, although the user may press the previous icon 332 to proceed to a prior stage of the procedure.

As shown in FIG. 3C, the primary image 312 comprises a transverse ultrasound image such as transverse image from TRUS probe 150, and the secondary image 314 comprises the endoscope image such as cystoscope image. The one or more modes 340 comprises the setup mode and the one or more sub-modes 342 comprises the align mode with the align sub-mode highlighted in relation to the other sub-modes 342, such as the hand piece mode 343. One or more markers 303 shows a position of the display corresponding to an end of the ultrasound probe such as TRUS probe 160. In some embodiments, path of energy 306 from the energy source is visible on the primary image 312 shown on the display.

In some embodiments, the processor is configured with instructions to automatically change the primary image 312 shown on the display in accordance with the current task to be performed by the user. As shown in FIG. 3C, the UI has automatically changed the primary image 312 to a real time ultrasound transverse image from the real time longitudinal image as shown in FIG. 3B, while the secondary image 314 remains the real time endoscope image.

The one or more user instructions 304 are configured to instruct the user at this stage of the align sub-mode. In some embodiments, the user is instructed to press on the foot pedal to release energy from the energy source, in order to detect the position of the energy source such as a water jet, although other energy sources can be detected on the primary image as described herein. In some embodiments, the user is instructed to rotate the imaging device such as the TRUS probe about a longitudinal axis of the probe to position a hyperechoic shadow of the treatment probe at a desired position, such as 12 o'clock, which corresponds to a substantially vertical position above the imaging probe as shown in the image, e.g. to within about 10 degrees of vertical. In some embodiments, the user is instructed to provide an input to the foot pedal to visualize the position and orientation of the energy 306 from energy source.

In some embodiments, the user is instructed to rotate the handpiece about the longitudinal axis of the probe and the handpiece, for example to rock the handpiece, such that the orientation of the energy source such as a water jet comprises a substantially vertical orientation, e.g. to within about 10 degrees of vertical, although any suitable orientation can be used. In some embodiments, the positioning of the display above the probes facilitates alignment of the probes, for example with the active light emitting area of the display oriented such that the display is substantially parallel to the transverse image and the longitudinal axis of one or more of the probes substantially perpendicular to the active light emitting area of the display as described herein. In some embodiments, the user is instructed to provide an input such as a click or touch on the information icon 328 to obtain additional information about the current mode and sub-mode, for example. In some embodiments, the user is instructed to change the view from the imaging device from a longitudinal mode such as a sagittal mode to a transverse mode.

In some embodiments, the UI 103 is configured for the user to press the next icon 334 to proceed to the next stage of the procedure, although the user may press the previous icon 332 to proceed to a prior stage of the procedure.

Referring to FIG. 3D, the user interface 103 can be configured to receive user input to plan the treatment with a plurality of images that are transverse to the probe 150, such as a plurality of transverse images from the TRUS probe. In some embodiments, the mode of the one or more modes 340 comprises a plan mode. In some embodiments, the plan mode comprises a plurality of sub-modes 342, in which the plurality of sub-modes comprises an angle and depth mode, a registration mode, and a profile mode. As shown in FIG. 3D, the plurality of modes 340 comprises the plan mode and the plurality of sub-modes 342 comprises an angle and depth mode 346. In some embodiments, the UI is configured to highlight the current mode such as the plan and the sub-mode such as the angle and depth mode 346. With the angle and depth sub-mode 346, the user is able to adjust the angle of treatment and the radial distance of the treatment from the probe such as a depth of treatment from the probe 150.

With the plan mode and the angle and depth sub-mode, the user is provided with an instruction 304 to select an anatomical tab and adjust the plurality of markers 353.

In some embodiments, in the plan mode the user interface 103 is configured for the user to provide input related to the angles of treatment delivered with the energy source such as a directional energy source on the treatment probe 150. In some embodiments, the primary image 312 comprises a transverse image such as a transverse ultrasound image, and the secondary image 314 comprises an endoscope image such as a cystoscope image. The image display area 310 shows the primary image and the secondary image. A plurality of markers 353 is, shown overlaid on the primary image. The plurality of markers 353 is configured to allow the user to adjust the treatment.

In some embodiments, the primary image 312 shown on the display comprises an image among a plurality of images at different tissue depths, and the user interface 103 is configured for the user to select the primary image to display among a plurality of images along image planes from different depths of the tissue, such as transverse images from different longitudinal locations along probe 150. A plurality of planning tabs 390 is shown above the primary image shown on the display, which allows the user to select the image to view for planning the treatment, although the plurality of planning tabs may be displayed at other locations. The plurality of planning tabs may comprise a first tab corresponding to a first depth along a longitudinal axis of the probe, such as a median lobe of the prostate, a second tab corresponding to a second longitudinal depth along the probe, such as a bladder neck, and a third tab corresponding to a second longitudinal depth along the probe, such as a mid-prostate, for example. The plurality of markers 353 is shown on each of the plurality of images selected with a corresponding tab.

In some embodiments, the plurality of markers 353 are shown at initial positions for each of the plurality of planning tabs and subsequently adjusted by the user. The initial positions of the plurality of markers may comprise default values, or values suggested with an artificial intelligence (AI) algorithm. In some embodiments, the UI is configured to display an assisted planning icon 399 for the user to turn on assisted planning. In some embodiments, the assisted planning is configured to indicate one or more tissue boundaries with a marker, such a dashed line. In some embodiments, the tissue boundary comprises a prostate boundary such as a boundary of the prostate. Alternatively or in combination, the assisted planning may provide initial locations for the plurality of markers 353 on the display, in which the locations of the plurality of markers 353 correspond to locations determined with the AI algorithm.

In some embodiments, a reset input 396 is shown with an icon and configured for the user to reset the markers shown in the currently selected tab to previous locations that the user has entered, a default locations, or locations determined with the AI algorithm.

The UI is configured for the user to plan the treatment at a plurality of depths with the markers 353 shown on each of the plurality of images corresponding to different depths in the tissue. This allows the user to plan the treatment angles at different depths of the tissue. In some embodiments, the user selects the planning tab corresponding to an image, and adjusts the locations of the one or more markers 353 on the image shown with the corresponding planning tab.

The UI 103 is configured for the user to select a marker among the plurality of markers 353 to adjust the location of the selected marker on the primary image 312. In some embodiments, the UI comprises a plurality of marker select icons such as buttons 350, which allows the user to select a marker to adjust among the plurality of markers 353. In some embodiments, the plurality of markers 353 comprises a first marker (1), a second marker (2), and a fourth marker (4). With a marker selected, the user is able to adjust the position of the selected marker on the primary image 312, which results in a corresponding change to one or more of the depth of treatment from the probe or the angular location of the treatment. The selected marker among the plurality of markers can be moved with user input in any way. In some embodiments, the display comprises a touch screen display, in which the UI 103 is configured for the user to drag the marker to a desired location. Alternatively or in combination, the marker can be moved with an input to directional icon, such as directional pad 374, for example. In some embodiments, the UI is configured for the user to make a coarse adjustment to the selected marker shown on the display by dragging the marker to a different location on the touch screen display, and to make a fine adjustment by pressing a button on the directional icon such as directional pad 374. In some embodiments, the directional pad is configured to move the location of the selected marker by an incremental amount in response to the user depressing a directional icon, such as an amount within a range from about 0.05 mm to about 0.2 mm in response to the user pressing on the directional icon, for example. In some embodiments, the amount of movement corresponds to a scale of the image shown on the display, such as a scale in millimeters (mm).

As shown in FIG. 3D, for example, marker three (3) has been selected and the user is allowed to adjust the position of marker three. The selected marker can be highlighted on the display, for example with a color, or other indication such as geometric shape around the marker. The geometric shape may comprise any suitable shape such a bracket, circle, square or diamond, for example.

In some embodiments, the UI 103 is configured is configured to constrain the direction of movement of the selected marker and to indicated to the user that the direction of movement of the marker has been constrained, for example by highlighting the allowable directions on the icon. As shown in FIG. 3D, the movement of the selected marker among the plurality of markers is limited to up and down movement on the display, and the directional icon such as direction pad 374 shows the up and down arrows highlighted with respect to the lateral arrows.

In some embodiments, the UI 103 is configured for the user to select each of the plurality of markers, e.g. markers 1, 2, 3 and 4, and to adjust the position of each of the selected markers. For example, the user can select marker 1 to align the treatment profile with the image of the treatment probe 150 shown in the primary image 312. In some embodiments, the directional icon such as directional pad 374 shows four directional icons highlighted, e.g. left, right, up and down, to indicate to the user that the marker can be moved in each of the corresponding directions. The user can select marker 2 to adjust the depth of the treatment. The user can select marker 3 to adjust an angle of treatment and a depth of the treatment with respect to treatment probe 150 corresponding to the position of marker 1. In some embodiments, the directional icon such as directional pad 374 shows four directional icons highlighted, e.g. left, right, up and down, to indicate to the user marker 3 can be moved in each of the corresponding directions. The user can select marker 4 to adjust an angle of treatment and a depth of the treatment with respect to treatment probe 150 corresponding to the position of marker 1. In some embodiments, the directional icon such as directional pad 374 shows four directional icons highlighted, e.g. left, right, up and down, to indicate to the user marker 4 can be moved in each of the corresponding directions. The adjustment to markers 3 and 4 can be used to adjust the planned angle of treatment 392, which corresponds to an angle between marker 3 and marker 4. The planned angle of treatment 392 can be shown on the display, and may comprise any suitable angle, such that the user can adjust the displayed angle of treatment 392 from 145.5 degrees to another angle in response to user preferences.

If the user is not satisfied with a position of the selected marker after adjustment, the user can select the undo icon 351 with a user input such as touching the display at the location of the undo icon, which will move the marker to a location of the marker prior to user adjustment.

In some embodiments, the UI 103 is configured for the user to press the next icon 334 to proceed to the next stage of the procedure, although the user may press the previous icon 332 to proceed to a prior stage of the procedure.

Referring to FIG. 3E, the user interface can be configured to receive user input to plan the treatment with one or more longitudinal images such as sagittal images. The one or more longitudinal images is aligned with treatment probe 150, such that treatment probe 150 extends a distance along the one or more longitudinal images. The one or more longitudinal images may be acquired with the TRUS probe, an ultrasound probe located on the treatment probe 150, or an external probe coupled to a skin of the patient, for example. As shown in FIG. 3E, the plurality of modes 340 comprises the plan mode and the plurality of sub-modes 342 comprises the profile mode 344. In some embodiments, the UI is configured to highlight the current mode such as the plan mode and the sub-mode such as the profile sub-mode 344. With the profile sub-mode 344 highlighted, the user is able to adjust the distance from the treatment probe, such as a depth of treatment from the probe 150.

In some embodiments, in the plan mode the user interface 103 is configured for the user to provide input related to the depth and longitudinal position of treatment delivered with the energy source such as a directional energy source on the treatment probe 150. In some embodiments, the primary image 312 comprises the longitudinal image such as a sagittal ultrasound image, and the secondary image 314 comprises an endoscope image such as a cystoscope image. A plurality of markers 353 is, shown overlaid on the primary image. The plurality of markers 353 is configured to allow the user to adjust the treatment.

In some embodiments, the UI 103 is configured for the user to perform a registration step at a registration stage of the work flow. The registration step may be performed as part of the planning mode of the one or more modes 340. In some embodiments, the user performs the registration step at a registration sub-mode of the one or more sub-modes 342. In some embodiments, the registration sub-mode is performed between the angle and planning sub-mode and the profile sub-mode, although the registration can be performed at any suitable stage of the procedure, for example with the alignment stage.

In some embodiments, the user views the treatment probe with a longitudinal image such as a sagittal image, such that the treatment probe 150 extends along a plane of the image. With the registration sub-mode, the user indicates a location of the energy source with a user input with the energy source located at a distal position. In some embodiments, the user inputs a second location corresponding to a proximal location of the energy source. In some embodiments, the user inputs the proximal location on the display. While the proximal location of the energy source can be input in many ways, in some embodiments the user places a marker along a pathway of the energy source, for example at a location along the probe. Alternatively or in combination, the user can move the energy source to the proximal location and input the proximal location. In some embodiments, the energy source such as a water jet is activated at the first location to allow the user to accurately identify the position of the energy source shown on the display and provide an appropriate input at the location. In some embodiments, the first location and the second location are shown with a line extending therebetween, and the user is allowed to adjust one or more of the first location or the second location with a coarse or fine input to the display, for example with the directional pad 374, for example. The first location and the second location can be used to register the plurality of transverse images with the position of the probe, which can be helpful for generating a treatment plan such as a 3D treatment plan.

As shown in FIG. 3E, the treatment probe 150 is shown on the image with a plurality of markers 353. In some embodiments, a longitudinal scale 380 is shown with a radial scale from the longitudinal axis of the probe such as a depth scale 382. The primary image 312 is with a plurality of markers 353 for the user to adjust the treatment on the longitudinal image. In some embodiments, one or more treatment limits 355 is show on the display to show the maximum depth of treatment. In some embodiments, the plurality of markers shown on the display correspond to plurality of regions of tissue to be protected. The markers may correspond to any tissue, such a verumontanum (veru), which can be identified veru protection zone (VPZ) marker 362, or a bladder neck (BN) identified with a BN marker 364. Each of the plurality of protection zones can be adjust with a user input to a corresponding icon. In some embodiments, the treatment at the VPZ can be adjusted with an input to a VPZ icon 366. In some embodiments, the treatment at the BN can be adjusted with an input to a BN icon 368.

With the plan mode and the profile sub-mode, the user is provided with an instruction 304 to adjust the plurality of markers 353 as needed for treatment.

In some embodiments, the UI 103 is configured with the plurality of selection markers 350, in which a user can select a marker to be adjusted as described herein. The selected marker can be adjusted with coarse or fine adjustment as described herein. In some embodiments, the starting location of the treatment is selected for adjustment with a treatment start (TS) icon 352. Once the TS icon 352 has been selected, the location of the start of the treatment can be adjusted with coarse or fine adjustment as described herein. In some embodiments, the ending location of the treatment is selected for adjustment with a treatment end (TE) icon 354. Once the TE icon 354 has been selected, the location of the end of the treatment can be adjusted with coarse or fine adjustment as described herein. In some embodiments, the user interface 103 comprises a plurality of user selectable markers 353, such as a first selectable marker (1), a second selectable marker (2), a third selectable marker (3), a fourth selectable marker (4) and a fifth selectable marker (5). Each of the plurality of markers can be selected with a user input to a corresponding icon among the plurality of icons 350, and the location of the corresponding marker adjusted on the primary image 312, for example with coarse or fine movement as described herein. For example a first marker can be selected with a first input to a first icon 356, such as a user touching the first icon 356. Additional markers can be selected and adjusted as described herein.

As shown in FIG. 3E, for example, marker four (4) has been selected and the user is allowed to adjust the position of marker four, for example with coarse or fine adjustment as described herein. The selected marker can be highlighted on the display, for example with contrast, a color, or other indication such as geometric shape around the marker as described herein. The geometric shape may comprise any suitable shape such a bracket, circle, square or diamond, for example. The directional icon such as directional pad 374 can be highlighted to indicate the allowable movements of the marker, for example.

The plurality of treatment markers shown in the plurality of transverse images and the one or more longitudinal images such as one or more sagittal images can be used to generate a three-dimensional treatment plan.

In some embodiments, the UI 103 is configured for the user to press the next icon 334 to proceed to the next stage of the procedure, although the user may press the previous icon 332 to proceed to a prior stage of the procedure.

Referring to FIG. 3F, the UI 103 is shown in a treatment mode. A treat mode 347 is shown highlighted among the plurality of modes 340, and the treatment sub-mode is show highlighted. The primary image is shown on the display with a locked icon 397 to indicate that the treatment plan has been accepted and locked. The primary image and the secondary image are shown on the display as described herein. The user instruction 304 shows an instruction for the user to begin treatment by pressing and holding the foot pedal and to pause treatment by releasing the foot pedal. In some embodiments, the instruction comprises an instruction not to move the TRUS probe or the handpiece, for example. In some embodiments, the longitudinal image such as a sagittal image is shown as the primary image on the display. With the treatment mode, information related to the treatment is shown on the display, such as an intended position of the energy source along the longitudinal axis of the probe overlaid on the image. In some embodiments, the longitudinal scale 380 and the radial scale such as depth scale 382 are shown on the longitudinal image such as the sagittal image during treatment.

Although FIGS. 3A to 3F show a user interface in accordance with some embodiments, one of ordinary skill in the art will recognize many variations. For example, some of the screens may be modified, the order of the screens can be provided in sequence as shown or the order changed. Some of the screens may not be used and some of the screens can be modified in accordance with the present disclosure. Some of the modes may comprises sub-modes of other modes, and some of the sub-modes may comprises one or more modes with additional sub-modes.

The system 100 can be configured in many ways in accordance with the present disclosure. In some embodiments, the one or more processors of the system 100 is configured to provide one or more safety features. In some embodiments, the one or more processors is configured to prime the energy source initially at a first flow rate with a first source of saline prior to insertion of the treatment probe into the patient, and the processor is configured to decrease the flow rate to the energy source to prime the energy source a second time at a second flow rate less than the first flow rate with a second source of saline if the first source has been depleted with the treatment probe inserted into the patient. In some embodiments, the system is configured to prime the energy source such as a water jet emitted from the probe 150. In some embodiments the system is configured to prime the energy source at over 90% of the treatment flow rate initially, e.g. at 100% of the treatment flow rate and to provide instructions to the user to prime the water jet with the water jet outside the patient. If system runs low on saline or out of saline to the energy source after the initial priming and prior to completion of treatment, the one or more processors of the system 100 are configured to automatically reduces the priming of the energy source to no more than 80%, for example to 50% or less. While this safety feature can be provided in many ways, in some embodiments the system is configured to automatically reduce the priming flow rate if the system runs low on saline or out of saline after the probe such as the TRUS probe has been inserted into the patient.

In some embodiments, the treatment probe comprises a handpiece and a motor-pack, and the one or more processors is configured to detect decoupling of the motor-pack from the handpiece. The processor is configured to disable treatment and direct the user to repeat one or more of an alignment mode or a planning mode of treatment prior to allowing the user to resume treatment, for example. In some embodiments, if the motor-pack and handpiece have become disconnected, the system is configured to bring the user back to the handpiece step, e.g. the handpiece sub-mode of the plurality of modes, for the user to complete one or more previously completed steps such as alignment and the planning steps. In some embodiments, the user interface is configured force the user to complete one or more of these previously completed steps.

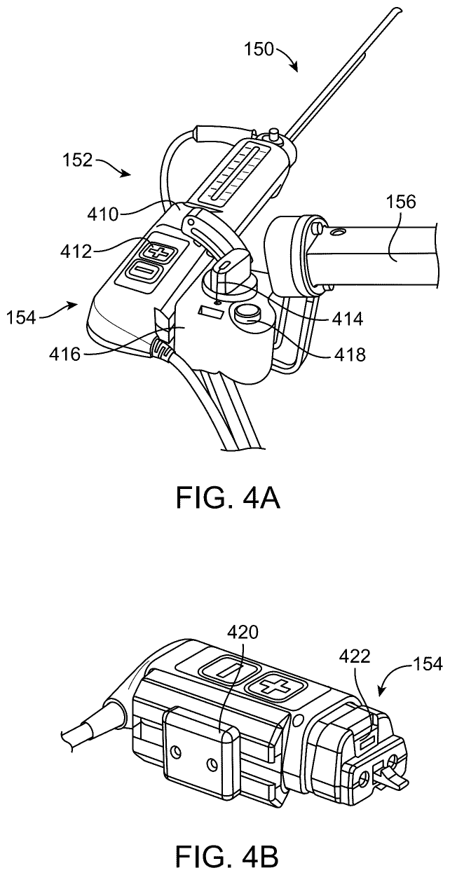

FIG. 4A shows a treatment probe 150 configured for use with a surgical system. The treatment probe 150 comprises a distal portion sized for insertion into the patient. In some embodiments, the treatment probe comprises a handpiece 152 configured to manipulate the probe during insertion of the distal end into the patient. In some embodiments, the handpiece 152 is configured to couple to one or more actuators such as motors configured to move the treatment probe in response to computer control as described herein. In some embodiments, the handpiece is configured to couple to a motor-pack 154, which is configured to move an energy source such as directional energy source to a plurality of positions and orientations as described herein. In some embodiments, the handpiece 152 comprises a handpiece release button 410 configured to release the handpiece 152 from the motor-pack 154. In some embodiments, the motor-pack comprises one or more controls 412 to control an amount of energy of the energy source, such as an amount of waterjet power to a water jet.

While the handpiece 152 can be configured in many ways, in some embodiments, the handpiece 152 comprises a linkage to rotate and translate the energy source in response to movements such as rotational movements transmitted from the motor-pack.

While the arm 156 that supports treatment probe 150 can be configured in many ways, in some embodiment the arm comprises lever and a motor-pack release knob 414 configured to release the motor-pack from arm 156, and to securely couple the motor-pack to the arm. While motor-pack 154 can be coupled to the arm 156 in many ways, in some embodiments, the arm 156 comprises a magnetic mount 416 configured to couple to the motor-pack to the arm. In some embodiments, the arm comprises a leveling button configured for a user to level the motor-pack, for example by pressing the button and then releasing the button to lock in the angle of inclination of the motor-pack, e.g. to level the motor-pack and the probe.

FIG. 4B shows the motor-pack 154 configured to couple to the handpiece 152 of treatment probe 150 and the arm 156. In some embodiments, the motor-pack 154 comprises a mounting plate 420 configured to couple to the arm. In some embodiments, the motor-pack 154 comprises a handpiece interface 422 configured to interface the motor-pack with the handpiece and may comprise one or more gears and an electrical connector to couple to the handpiece.

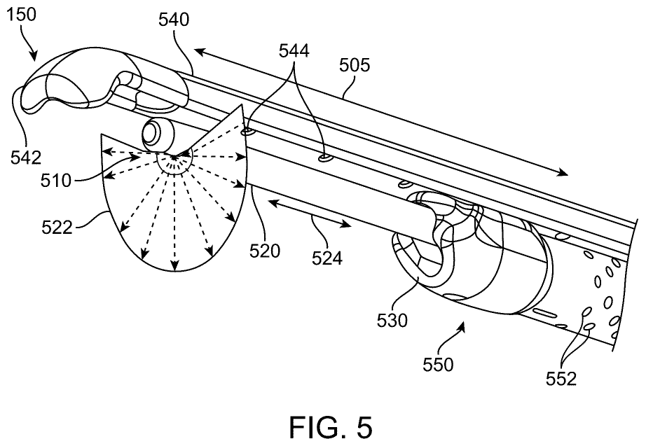

FIG. 5 shows treatment probe 150 and corresponding movements of an energy source 510. In some embodiments, the energy source 510 is carried on a carrier 520 that is configured to carry the energy source move the energy source. In some embodiments, the carrier 520 is configured to carry the energy source to a plurality of angles and longitudinal positions corresponding to a treatment plan such as a three dimensional treatment plan as described herein. In some embodiments, energy source 510 is configured to rotate to a plurality of angles 522 in accordance an angle of the treatment plan as described herein. In some embodiments, the energy source is configured to translate as shown with arrows 524. The rotational movement and translational movement of the energy source can be combined in accordance with the 3D treatment plan as described herein, for example.

The energy source 510 may comprise any suitable energy source, such as such as one or more of an electrode, a loop electrode, a laser source, a thermal energy source, a mechanical energy source, a mechanical sheer, an ultrasound probe, a cavitating ultrasound probe, a water jet, e.g. a fixed pressure water jet, a plasma source, a steam source, a morcellator, a trans urethral needle, a photo ablation source, a radiation energy source, a microwave energy source or a water jet evacuation source, for example.

While the treatment probe 150 can be configured in many ways, in some embodiments the treatment probe comprises an opening 530 configured to receive an endoscope such as a cystoscope to view the treatment area. In some embodiments, the probe 150 comprises a support portion 530 that is configured to support the carrier and the endoscope. The support portion 530 may comprise one or more openings 552 configured to release a fluid such as saline from a saline source as described herein. In some embodiments, the probe comprises a portion 540 comprising sufficient stiffness to advance the probe into the patient. In some embodiments, the stiff portion 540 extends to a curved distal end 542 to allow the probe to be inserted into tissue, such as along a lumen. In some embodiments, the stiff portion 540 comprises one or more openings 544 to remove material such as fluids from the surgical site. In some embodiments, the one or more openings 544 is coupled to an evacuation pump such as the aspiration pump as described herein.

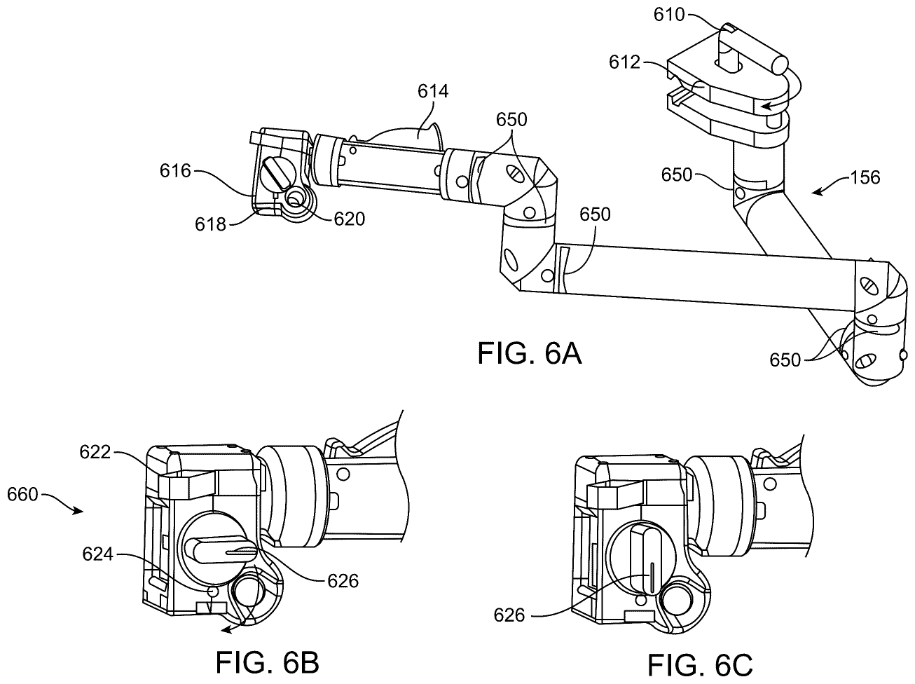

FIG. 6A shows an arm 156 configured to support the treatment probe 150. The arm comprises a bedrail clamp 612 configured to couple to a bedrail of the patient support as described herein. The arm 156 comprises a clamp lever 610 configured to securely couple the bedrail clamp 610 onto the bedrail of the patient support. In some embodiments, the arm comprises an articulation release lever 614 configured to allow the arm to articulate to adjust the position of the arm. The arm may comprise a plurality of locking joints 650 that are configured to move freely when the articulation release lever 614 is pressed down by a user, and then locked in place when the lever is released. The joints of the arm may comprise locking joints, such as joints with clutches, or magnetic joints, that can be moved into position prior to surgery and lock into place during surgery with computer controlled movement of the probe as described herein. Although reference is made to a manually manipulated arm, in some embodiments the arm comprises a robotic arm.

FIG. 6B shows mounting device 660 configured to couple to the motor-pack, in which the mounting device 660 comprises an unlocked configuration. The mounting device 660 comprises a rotation guide rail 622, a mounting lock indicator 624, and a motor-pack release knob 626. As shown in FIG. 6B, the release knob 626 is shown in an unlocked configuration.

FIG. 6C shows the mounting device of FIG. 6B, in which the release knob 626 comprises a locked configuration, in in which the motor-pack release knob has been rotated to a locked configuration.

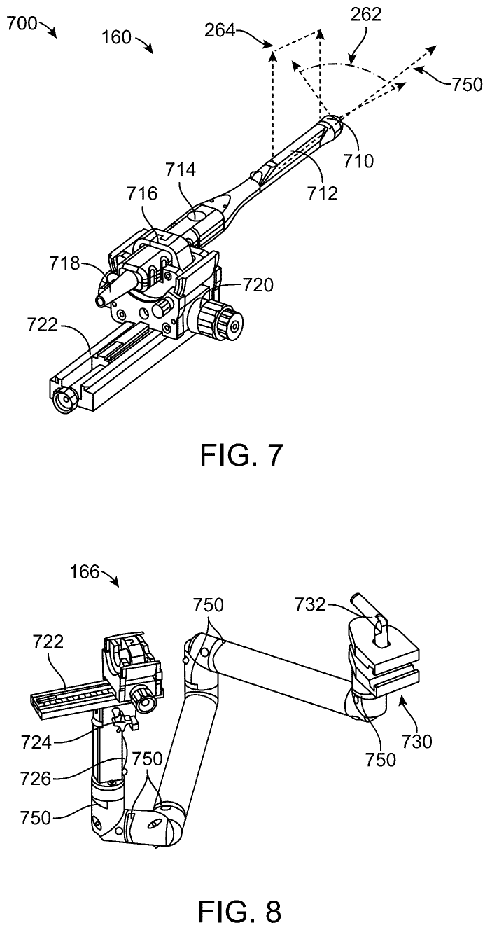

FIG. 7 shows an ultrasound imaging device 700 to image tissue of the patient. The imaging device comprises one or more arrays to image tissue of the patient. The imaging device 700 may comprise an ultrasound imaging array, and may comprise a one dimensional (1D), a two dimensional (2D) or a three dimensional (3D) imaging device. In some embodiments the one or more arrays comprises a transverse array 710 configured to image tissue along a plane that is transverse to an elongate axis of the probe 750. The probe 160 extends in an elongate direction along an axis 750 to a distal tip sized for inversion into a patient. The transverse array 710 extends in a direction transverse to the longitudinal axis 750 of the probe 160 to generate images that are transverse to the longitudinal axis 750, for example perpendicular to the axis 750. In some embodiments, the ultrasound imaging device is configured to generate transverse images within a field of view 262. In some embodiments, the ultrasound imaging device comprises a longitudinal array of transducers, such as a sagittal array 712 of transducers. The longitudinal array of transducers 712 extends in a longitudinal direction along probe 150 corresponding to axis 750. In some embodiments, the longitudinal array of transducers 712 is configured to generate a longitudinal image of tissue within a longitudinal image field of view, such as a sagittal plane image field of view 264.

In some embodiments, the ultrasound imaging device comprises a T-shaped marking to allow the user to identify the longitudinal axis of the array and the transverse axis of the array. In some embodiments, the transverse array 710 corresponds to a transverse imaging plane of the T-shaped marking, e.g. the top of the T-shaped marking, and the and the longitudinal array such as sagittal array 712 corresponds to the longitudinal imaging plane of the T-shaped marking, e.g. the bottom part of the T-shaped marking.

In some embodiments, the imaging device 700 comprises a probe configured to be inserted into the patient, such as a TRUS probe 160. In some embodiments, the TRUS probe 160 comprises plane switch button 714 to toggle between longitudinal and transverse images shown on the display as described herein. In some embodiments, the TRUS probe comprises a probe cradle and latch 716 configured to couple the probe 150 to a stepper 722. The stepper comprises a knob 720 that allows the stepper to advance and retract the probe from the patient. The stepper is configured to couple to the TRUS arm as described herein and move the probe 160 while the arm remains fixed, for example with rotation of the knob 720. By rotating the knob to a plurality of positions, the probe is placed at a plurality of locations corresponding to a plurality of depths of the transverse images, which can be used for treatment planning as described herein. In some embodiments, the TRUS probe 160 comprises a cable 718 extending from a proximal portion of the TRUS probe 160.

In some embodiments, the location of the one or more of the longitudinal array such as sagittal array 712 or the transverse array 710 of the ultrasound device 700 is visible to the user. In some embodiments, the one or more of the longitudinal array or the transverse array is covered with a material which is visibly different from other areas of the distal end of the probe, such as with a different color or shading for example. The visible portion of the probe corresponding to the locations of the one or more transducer arrays can be associated with the position of the display. In some embodiments, the transverse array is approximately parallel to an active light emitting area of the display, and the visible portion of the transverse array can help the user associate the physical location of the tissue shown in transverse images with the location and orientation of the transverse array 710. In some embodiments, the longitudinal array such as the sagittal array 712 is approximately perpendicular to an active light emitting area of the display, and the visible portion of the longitudinal array can help the user associate the physical location of the tissue shown in longitudinal images such as sagittal images with the location and orientation of the longitudinal array such as sagittal array 712. In some embodiments, the ultrasound imaging device comprises TRUS probe 160, in which the locations of transverse array 710 and the longitudinal array such as sagittal array 712 are visible to the user in relation to the longitudinal axis 750 of the probe, for example.

FIG. 8 shows an arm 166 configured to support an ultrasound imaging device as in FIG. 7. The arm comprises a bedrail clamp 730 is configured to couple to a bedrail of the patient support as described herein. The arm 166 may comprises a clamp lever 732 configured to securely couple the bedrail clamp 730 onto the bedrail of the patient support. In some embodiments, the arm comprises an articulation release lever 726 configured to allow the arm to articulate to adjust the position of the arm. The arm may comprise a plurality of locking joints 750 that are configured to move freely when the articulation release lever 726 is pressed down by a user, and then lock in place when the lever is released. The joints of the arm may comprise locking joints, such as joints with clutches, or magnetic joints, that can be moved into position prior to surgery and locked into place during surgery with computer controlled movement of the probe as described herein. Although reference is made to a manually manipulated arm, in some embodiments the arm comprises a robotic arm.

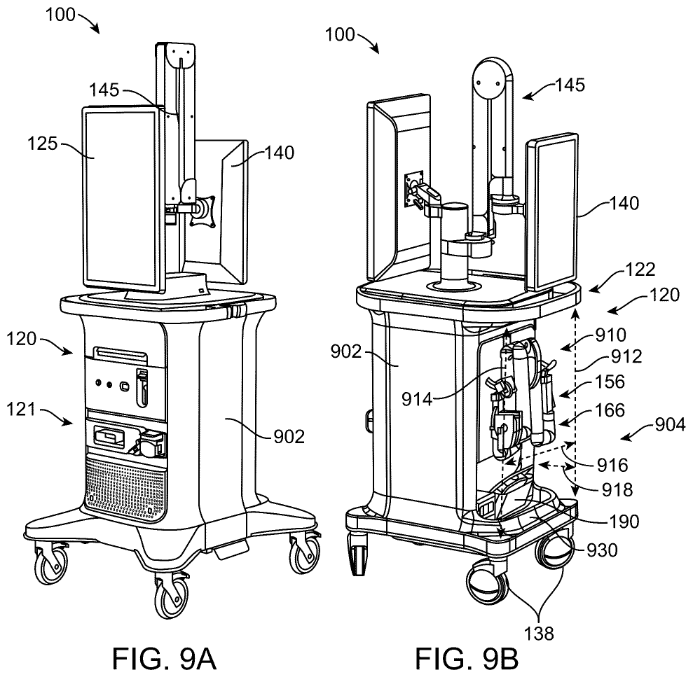

FIGS. 9A and 9B show a front side view and a rear side view, respectively, of the surgical system of FIGS. 1 and 2 in a compact configuration suitable for moving or storing the console, in accordance with some embodiments. The console 120 comprises a front side 121, a patient facing side 902, a rear side 904, and a side facing away from the patient. In the compact configuration display arm 145 comprises a compact configuration, in which the surgeon display 140 is placed within the lateral profile of console 120 to decrease size and increase the stability for mobility and transport of the surgical system. In some embodiments, the lateral profile of the console corresponds to a perimeter of the console at the height of the rail, and the display is 140 is positioned with the arm 145 in the compact configuration so as not to extend laterally beyond the perimeter of the console at the height of the rail. In some embodiments, the console display 145 is supported on the console so as not to extend laterally beyond the perimeter of the console at the height of the handle 122. In some embodiments, the front side 121 comprises the plurality of connectors and ports referred to in FIG. 1. The console 120 comprises a rear side 904 opposite the front side 121, a patient facing side 902, which faces the patient during surgery, and a fourth side opposite the patient facing side. The patient facing side 902 and the fourth side facing away from the patient are located between the front side 121 and the back side 904.

In some embodiments, the console 120 comprises a recess 910 configured to store one or more components of system 100 when the system is not in use. The recess 910 can be configured to store components of system 100 such as one or more of the foot pedal 190, the arm 156, the arm 166 and one or more power cables, for example. In some embodiments, the recess 910 comprises one or more features for storing the arms, such as one or more of hooks, fasteners, latches, clips or straps located to hang arm 156 and arm 166 in a compact configuration. In some embodiments, arms 156 and arms 166 comprise 6 joints connected with segments configured to fold into a compact configuration sized to fit into recess 910. In some embodiments, the recess 910 comprises a protrusion 930 extending upwardly to define a container portion sized and shaped to store one or more components such as the foot pedal, for example.

While the recess 910 can be sized and shaped in many ways, in some embodiments, the recess 910 comprises a height 912, a width 916 and a depth 918 sized to store the arms 156, 166 in the compact configuration, such that the arms 156, 166 do not extend laterally beyond handle 122. In some embodiments, the width 916 is defined by a distance between a height 912 of recess on a first side and a height 914 of the recess on a second side. In some embodiments, the heights 912, 914, correspond to a distance from a corner of handle 122 to lower corner portions of console 120 above wheels 138.

In some embodiments, the recess 910 is located on the rear side 904 of the console 120, although the recess 910 can be located on the patient facing side or the fourth side facing away from the patient, for example.

In order to configure system 100 for surgery, the arm 145 is opened and rotated toward the patient side 902 of the console to position display 140 above the patient as described herein.

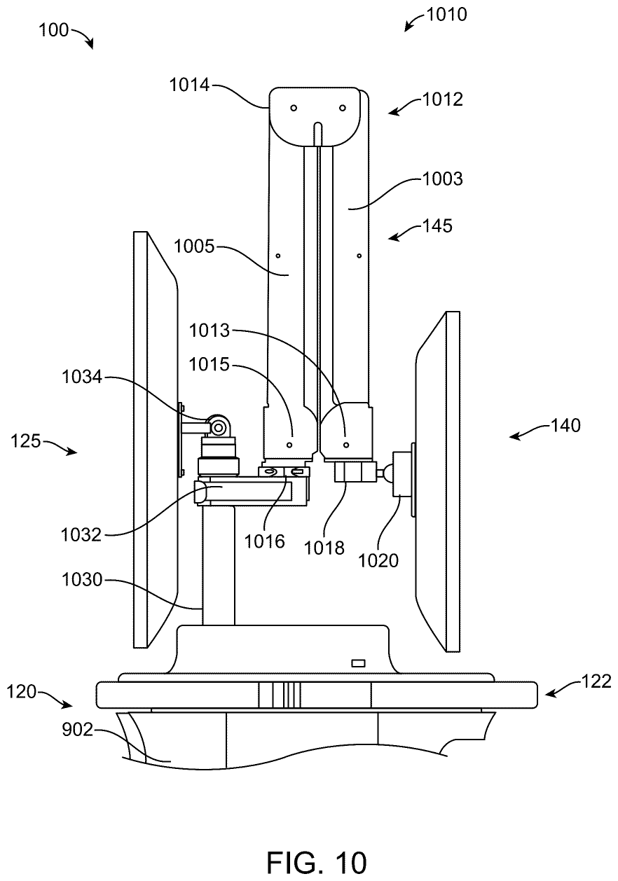

FIG. 10 shows side view of the surgical robotics system 100 in a compact configuration. In the view shown, the system 100 is viewed from the patient facing side 902 of the console 120. In this configuration, the console display 125 and the surgeon display 140 are located within the perimeter of the console at the height of the handle 122.

The console display 125 and the surgeon display 140 are supported with a plurality of components such as joints and segments to allow the displays to be adjusted during surgery and to be moved into the compact configuration for storage. The surgeon display 140, the arm 145, and the console display 125 are supported with a post 1030. The post 1030 extends from the top of console 120. The post 1030 extends to a lateral segment 1032, which is coupled to the arm 145 with a joint 1016. The post 1030 extends upwardly to joint 1034 configured to allow adjustment to display 125. The joint 1016 is configured to allow arm 145 to rotate to place monitor 140 above the patient as described herein.

In some embodiments the console 120 has a length, a width, and a height, and the surgeon display 140 and the arm 145 are configured to fit within the length and the width of the console in a stowed position with the arm in a stowed configuration. In some embodiments, the arm 145 is configured to allow movement of the surgeon display 140 along the midline of the patient from between the legs of a patient to over the torso of the patient, for example in an unlocked configuration.

In some embodiments, one or more of the console display 145 or the surgeon display 140 is pivotable between a downward facing position and an upward facing position.

The arm 145 comprises a plurality of joints that allows the arm to be extended to support the display 140 above the patient as described herein. The arm 145 comprises a first segment 1005 coupled to joint 1016 with a pivot joint 1015 which allows the first segment 1005 to swing down. In some embodiments, the joint 1016 has a clamping mechanism to limit the rotational movement, e.g. drift, of the arm 1010 relative to segment 1032 in order to resist the forces of the touchscreen in response to the user touching, e.g. tapping, the user interface of display 140 to provide input. In some embodiments, the clamping mechanism comprises one or more clamps or clutches to decrease movement of the first segment 1005 relative to segment 1032 at joint 1016. In some embodiments, the clamping mechanism has a dampening feature to allow for repositioning the arm while stabilizing the console display 125.

In some embodiments, the arm 145 comprises a bracket 1010, which is coupled to the first segment 1005 with a joint 1014. In some embodiments, the joint 1014 has a clamping mechanism to limit the rotation of joint 1014 and extension of segment 1005 relative to segment 1003 during transport. The arm 145 comprises a second segment 1003, which is coupled to bracket 1010 with a joint 1012. The joint 1012 allows the second segment 1003 to swing outwardly to support the display 140 above the patient. The second segment 1003 comprises a second joint 1013, which is coupled to a joint 1018. The joint 1013 allows the joint 1018 to pivot with respect to second segment 1003, which facilitates placement of display 140. The joint 1018 allows rotation of the display relative to segment 1003. The joint 1018 is coupled to a gimbal 1020, which allows two dimensional rotation of display 140. {

In some embodiments, the first segment 1005 and the second segment 1003 comprise approximately equal lengths, e.g. to within 10% of each other, which helps to maintain a height of display 140 at approximately the same height in the compact configuration as the height when placed above the patient. In some embodiments the approximately equal lengths of the segments allows the 145 arm to extend to a maximum reach to from the console 120 and facilitate placement of the display 140 over the patient.

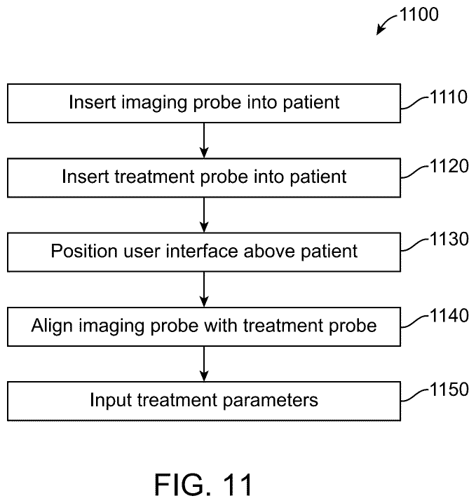

FIG. 11 shows a method 1100 of treating a patient.

At a step 1110, an imaging probe is inserted into the patient. The imaging probe may comprise any suitable imaging probe as described herein.

At a step 1120, the treatment probe is inserted into patient. The treatment probe may comprise any suitable treatment probe as described herein.

At a step 1130, the user interface is positioned above patient. The user interface may comprise any suitable interface as described herein. The user interface can be positioned above the midline of the patient, for example.

At a step 1140, the imaging probe is aligned with treatment probe. The imaging probe can be aligned with the patient with one or more of the modes and sub-modes as described herein, for example.

At a step, 1150, treatment parameters are input into the user interface with the user interface located above the patient. The treatment parameters may comprise any suitable input parameters provided with a user interface as described herein.

Although FIG. 11 shows a method of treating a patient in accordance with some embodiments, one of ordinary skill in the art will recognize many adaptations and variations. For example, the steps can be performed in sequence, or in a different order than shown. Some of the steps can be omitted and some of the steps repeated. Some of the steps may comprises sub-steps of other steps.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The “user interface” (UI) as described herein may comprise one or more components of UI as will be understood by one of ordinary skill in the art in computer science. The UI may comprise one or more components of a graphical user interface (GUI), and the GUI may comprise one or more input devices such as a display and one or more pointing devices such as touch pads, directional pads, push buttons, track pads, mice, joy sticks configured for a user to input commands to the computing device in association with the display. In some embodiments, the display comprises a touch screen display configured for the user to provide touch inputs to the computing device with the display. Alternatively or in combination, the UI may comprise voice commands or gesture sensing to allow the user to provide input such as input commands to the computing device. The processor can be configured to provide one or more features such as icons or controls on a display and to receive user input associated with the one or more features such as icons as described herein.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

As used herein, the term “e.g.” means for example.

The present disclosure includes the following numbered clauses.

Clause 1. A system to perform surgery on a patient, the system comprising: a console; one or more processors configured to couple to a treatment probe and an imaging device to control movement of the treatment probe and generate images of the tissue treated with the probe; a positioning arm coupled to the console; and a user interface comprising a display coupled to the positioning arm, the positioning arm configured to support the display at a first location above a midline of the patient with a first configuration and a second location away from the midline of the patient with a second configuration, the user interface configured to receive user a plurality of user inputs with the arm in the first configuration and the display supported above the midline of the patient.

Clause 2. The system of any of the preceding clauses, wherein the imaging device comprises an ultrasound imaging device configured to be inserted into the patient, and the arm is configured to support the display above the ultrasound imaging device with the ultrasound imaging device inserted into the patient.

Clause 3. The system of any of the preceding clauses, wherein the ultrasound imaging device comprises a transducer array to generate a transverse image along a transverse image plane, the arm configured to support the display with transverse image displayed on the display above the patient, the transverse image shown on the display approximately parallel to the transverse image plane.

Clause 4. The system of any of the preceding clauses, wherein the transverse image plane is parallel to the image shown on the display to within 30 degrees.

Clause 5. The system of any of the preceding clauses, wherein the ultrasound imaging device comprises an ultrasound array configured to generate a longitudinal image along a longitudinal image plane, the arm configured to support the display with longitudinal image displayed on the display above the patient.

Clause 6. The system of any of the preceding clauses, wherein the longitudinal image plane corresponds to a plane extending from the ultrasound transducer array and through a portion of the display.

Clause 7. The system of any of the preceding clauses, wherein the transducer device is rotatable about a longitudinal axis in order to rotate an angle of the longitudinal image plane to view the treatment probe with the plane extending through the transducer array, the treatment probe and the monitor.

Clause 8. The system of any of the preceding clauses, wherein the treatment probe extends through at least a portion of the longitudinal image plane.

Clause 9. The system of any of the preceding clauses, wherein the longitudinal image shown on the display is approximately perpendicular to the longitudinal image plane and optionally to within 30 degrees of perpendicular.

Clause 10. The system of any of the preceding clauses, wherein the ultrasound imaging device comprises a transrectal ultrasound (TRUS) imaging probe configured to view the treatment probe with the treatment probe located between the TRUS probe and the display.

Clause 11. The system of any of the preceding clauses, wherein the ultrasound imaging device comprises a transverse array and a longitudinal array, and the arm is configured to position the image shown on the display to within 30 degrees of parallel to a long axis of the longitudinal array and to within 30 degrees of perpendicular to a long axis of the transverse array.

Clause 12. The system of any of the preceding clauses, further comprising a patient support configured to support the patient with the patient support below patient, the midline of the patient corresponding to a plane extending through the display and the support.

Clause 13. The system of any of the preceding clauses, wherein the support comprises a plurality of stirrups to support legs of the patient splayed and feet of the patient above a torso of the patient, and wherein the display is sized to fit between knees of the patient.

Clause 14. The system of any of the preceding clauses, wherein the arm is configured to support the display above the patient and superiorly to a penile fenestration of a drape covering the patient.

Clause 15. The system of any of the preceding clauses, wherein the positioning arm is coupled to a top of the console.

Clause 16. The system of any of the preceding clauses, wherein the display comprises a touchscreen display.

Clause 17. The system of any of the preceding clauses, wherein the display is movable with the arm from the first location above the midline of the patient to the second location in which the second location comprises a stowed position above the console with the arm in the second configuration.

Clause 18. The system of any of the preceding clauses, wherein the console has a length, a width, and a height, and wherein the display and the arm are configured to fit within the length and the width of the console in a stowed position with the arm in the second configuration.

Clause 19. The system of any of the preceding clauses, wherein the touchscreen display is movable along the midline of the patient from between the legs of a patient to over the torso of the patient.

Clause 20. The system of any of the preceding clauses, wherein the touchscreen display is pivotable between a downward facing position and an upward facing position.

Clause 21. The system of any of the preceding clauses, wherein the arm comprises a plurality of joints and one or more of a locking arm, a clutch arm, a manual arm, a gas-spring arm, a robotic arm, a zero-gravity arm or a zero gravity robotic arm.

Clause 22. The system of any of the preceding clauses, wherein the gas spring arm comprises a gas strut and a spring.

Clause 23. The system of any of the preceding clauses, wherein the display comprises a touch screen display configured to receive a plurality of user inputs through a drape placed over the touch screen display and wherein the arm is configured to resist movement in the first configuration in response to the user input through the drape.

Clause 24. The system of any of the preceding clauses, the touchscreen display is configured to receive a plurality of user inputs from a finger of a user covered with a glove transmitted from the finger through the glove and the drape to the touchscreen display and wherein the arm is configured to resist movement in response to the user input transmitted through the glove and the drape and optionally wherein the drape comprises a sterile drape and the glove comprises a sterile glove.

Clause 25. The system of any of the preceding clauses, wherein the arm comprises a locking arm configured to lock the arm in place in the first configuration.

Clause 26. The system of any of the preceding clauses, wherein the positioning arm is configured to resist movement of the touchscreen display when placed above the midline and touched by a user with a first amount of force to input data and to allow movement of the display with a second mount of force greater than the first amount of force.

Clause 27. The system of any of the preceding clauses, wherein the first amount force is within a range from about 0.01 pounds to about 5 pounds and optionally within a range from about 0.01 pounds to about 2 pounds.

Clause 28. The system of any of the preceding clauses, wherein the second amount of force is greater than about 2 pounds and optionally greater than about 5 pounds.

Clause 29. The system of any of the preceding clauses, wherein the arm is configured with stiction to inhibit movement of the display when the first amount of force is applied touch screen when the touch screen is positioned above the midline of the patient and to allow movement with the second amount of force greater than the first amount of force.

Clause 30. The system of any of the preceding clauses, wherein movement of the display is inhibited with a locking mechanism.

Clause 31. The system of any of the preceding clauses, where the touchscreen display is coupled to the arm with the gimbal mount.

Clause 32. The system of any of the preceding clauses, wherein the user interface comprising the display has a height and a width, wherein the height is greater than the width.

Clause 33. The system of any of the preceding clauses, wherein the positioning arm is configured to align a vertical centerline of the display with a midline of the patient and a corresponding midline of the patient support.

Clause 34. The system of any of the preceding clauses, wherein the positioning arm is configured to place a vertical centerline of the display within about 2 inches of a midline of the patient with the patient on the support.

Clause 35. The system of any of the preceding clauses, wherein the positioning arm is configured to place a centerline of the touchscreen display to within 2 inches of a centerline of the treatment probe.

Clause 36. The system of any of the preceding clauses, wherein a second display is located above the console.

Clause 37. The system of any of the preceding clauses, wherein display comprises a touchscreen display and the one or more processors is configured to: generate, on the touchscreen display, an image display area to display one or more images, a control panel area comprising a plurality of input features to receive a plurality of a user inputs to control movement of the treatment probe, the control panel area below image display area.

Clause 38. The system of any of the preceding clauses, wherein the system comprises a plurality of modes, the plurality of modes comprising a setup mode, a planning mode and a treatment mode.

Clause 39. The system of any of the preceding clauses, wherein the user interface is configured for the user to touch the touchscreen display a total number of times at a plurality of locations to set up the treatment, plan the treatment and perform the treatment, and wherein at least half of the plurality of locations touched the total number of times is located below a centerline of the display.

Clause 40. The system of any of the preceding clauses, wherein the plurality of input features is configured to receive the plurality of user inputs to plan a treatment of the patient with the plurality of user inputs located below the image display area.

Clause 41. The system of any of the preceding clauses, wherein the control panel area comprises a plurality of user input features below the image display area to control the one or more images shown in the image display area.

Clause 42. The system of any of the preceding clauses, wherein the control panel area is configured to be touched by the user for each of the plurality of inputs below a centerline of the touchscreen display.

Clause 43. The system of any of the preceding clauses, wherein the imaging device comprises an ultrasound imaging probe and an endoscope camera and the one or more images comprises an ultrasound image and an endoscope image and the control panel area comprises a plurality of inputs located below the image display area to control the display of ultrasound image and the endoscope image.

Clause 44. The system of any of the preceding clauses, wherein the control panel area comprises a toggle input to toggle between the TRUS image and the cystoscope image within the image display area.

Clause 45. The system of any of the preceding clauses, wherein the image display area comprises a primary image and a secondary image and wherein the toggle input is configured to toggle between the primary image between the ultrasound image and the endoscope image.

Clause 46. The system of any of the preceding clauses, wherein the plurality of features to receive a plurality of user inputs below the image display area comprises one or more of an input to change an imaging plane of the ultrasound imaging probe, an image setting input to adjust a plurality of settings of the ultrasound device and an endoscope, an energy source control to control a longitudinal position of an energy source, a scale to measure a distance on an image, an information input to provide additional information related to the treatment, an assisted planning input to activate assisted treatment planning, an input to select a treatment plane, or a directional pad to adjust a location of treatment.

Clause 47. The system of any of the preceding clauses, wherein the processor is configured to generate a plurality of treatment markers overlaid on an image in the image display area, the plurality of treatment markers corresponding to one or more of a depth of treatment from the treatment probe, an angle of treatment relative to the treatment probe, or a longitudinal position along the treatment probe and wherein the control panel comprises a plurality of marker select icons configured to select a corresponding treatment marker of the plurality of markers to adjust a location of the corresponding treatment marker on the display.

Clause 48. The system of any of the preceding clauses, wherein user interface comprises a coarse adjustment and a fine adjustment of the selected treatment marker, the coarse adjustment configured for a user to adjust a location of the treatment marker on the display by placing a finger of the user on the treatment marker and sliding the treatment marker with the finger placed thereon to move the treatment marker to a second location on the image, the fine adjustment configured for the user to press a directional icon of a directional pad located in the control panel area.

Clause 49. The system of any of the preceding clauses, further comprising a foot pedal coupled to the processor and configured to control the activation of the treatment probe, the foot pedal configured to be located between lower limbs of the patient and optionally between feet of the patient.

Clause 50. The system of any of the preceding clauses, wherein the one or more processors is configured to prime the energy source initially at a first flow rate with a first source of saline prior to insertion of the treatment probe into the patient and wherein the processor is configured to decrease the flow rate to the energy source to prime the energy source a second time at a second flow rate less than the first flow rate with a second source of saline if the first source has been depleted with the treatment probe inserted into the patient.

Clause 51. The system of any of the preceding clauses, wherein the treatment probe comprises a handpiece and a motor-pack, and wherein the one or more processors is configured to detect decoupling of the motor-pack from the handpiece and wherein the processor is configured to disable treatment and direct the user to repeat one or more of an alignment mode or a planning mode of treatment prior to allowing the user to resume treatment.

Clause 52. The system of any of the preceding clauses, wherein the treatment probe and the imaging device are coupled to the console in electronic communication.

Clause 53. The system of any of the preceding clauses, wherein the treatment probe is electrically and fluidically coupled to the console.

Clause 54. The system or method of any of the preceding clauses wherein the longitudinal image comprises a sagittal image.

Clause 55. A method of preparing a patient for surgery, the method comprising: inserting an imaging probe into the patient; inserting a treatment probe into the patient above the imaging probe; positioning a user interface comprising a display above a midline of the patient; and inputting a plurality of treatment parameters into the user interface with the display positioned above the midline of the patient.

Clause 56. The system or method of any one of the preceding clauses wherein the imaging device comprises one or more of an ultrasound imaging device, a transrectal ultrasound imaging device, a doppler ultrasound imaging device, an endoscope, a cystoscope, a fluoroscopic imaging device, a C-arm imaging device, a fluoroscopic C-arm imaging device, a magnetic resonance imaging device, or a real time magnetic resonance imaging device.

Clause 57. The system or method of any one of the preceding clauses wherein the energy source comprises on or more of an electrode, a loop electrode, a laser source, a thermal energy source, a mechanical energy source, a mechanical sheer, an ultrasound probe, a cavitating ultrasound probe, a water jet, e.g. a fixed pressure water jet, a plasma source, a steam source, a morcellator, a trans urethral needle, a photo ablation source, a radiation energy source, a microwave energy source or a water jet evacuation source.

Clause 58. The system or method of any one of the preceding clauses wherein the endoscope comprises a cystoscope.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A system to perform surgery on a patient, the system comprising:
a console;
one or more processors configured to couple to a treatment probe and an imaging device to control movement of the treatment probe and generate images of the tissue treated with the probe;
a positioning arm coupled to the console; and
a user interface comprising a display coupled to the positioning arm, the positioning arm configured to support the display at a first location above a midline of the patient with a first configuration and a second location away from the midline of the patient with a second configuration, the user interface configured to receive user a plurality of user inputs with the arm in the first configuration and the display supported above the midline of the patient.

2. The system of claim 1, wherein the imaging device comprises an ultrasound imaging device configured to be inserted into the patient, and the arm is configured to support the display above the ultrasound imaging device with the ultrasound imaging device inserted into the patient.

3. The system of claim 2, wherein the ultrasound imaging device comprises a transducer array to generate a transverse image along a transverse image plane, the arm configured to support the display with transverse image displayed on the display above the patient, the transverse image shown on the display approximately parallel to the transverse image plane.

4. The system of claim 3, wherein the transverse image plane is parallel to the image shown on the display to within 30 degrees.

5. The system of claim 2, wherein the ultrasound imaging device comprises an ultrasound array configured to generate a longitudinal image along a longitudinal image plane, the arm configured to support the display with longitudinal image displayed on the display above the patient.

6. The system of claim 5, wherein the longitudinal image plane corresponds to a plane extending from the ultrasound transducer array and through a portion of the display.

7. The system of claim 6, wherein the transducer device is rotatable about a longitudinal axis in order to rotate an angle of the longitudinal image plane to view the treatment probe with the plane extending through the transducer array, the treatment probe and the monitor.

8. The system of claim 7, wherein the treatment probe extends through at least a portion of the longitudinal image plane.

9. The system of claim 5, wherein the longitudinal image shown on the display is approximately perpendicular to the longitudinal image plane and optionally to within 30 degrees of perpendicular.

10. The system of claim 2, wherein the ultrasound imaging device comprises a transrectal ultrasound (TRUS) imaging probe configured to view the treatment probe with the treatment probe located between the TRUS probe and the display.

11. The system of claim 2, wherein the ultrasound imaging device comprises a transverse array and a longitudinal array, and the arm is configured to position the image shown on the display to within 30 degrees of parallel to a long axis of the longitudinal array and to within 30 degrees of perpendicular to a long axis of the transverse array.

12. The system of claim 1, further comprising a patient support configured to support the patient with the patient support below patient, the midline of the patient corresponding to a plane extending through the display and the support.

13. The system of claim 12, wherein the support comprises a plurality of stirrups to support legs of the patient splayed and feet of the patient above a torso of the patient, and wherein the display is sized to fit between knees of the patient.

14. The system of claim 13, wherein the arm is configured to support the display above the patient and superiorly to a penile fenestration of a drape covering the patient.

15. The system of claim 1, wherein the positioning arm is coupled to a top of the console.

16. The system of claim 1, wherein the display comprises a touchscreen display.

17. The system of claim 1, wherein the display is movable with the arm from the first location above the midline of the patient to the second location in which the second location comprises a stowed position above the console with the arm in the second configuration.

18. The system of claim 17, wherein the console has a length, a width, and a height, and wherein the display and the arm are configured to fit within the length and the width of the console in a stowed position with the arm in the second configuration.

19. The system of claim 17, wherein the touchscreen display is movable along the midline of the patient from between the legs of a patient to over the torso of the patient.

20. The system of claim 17, wherein the touchscreen display is pivotable between a downward facing position and an upward facing position.

21.-58. (canceled)
